(19) United States
(12) Patent Application Publication
(10) Pub. No.: US 2004/0086883 A1
(43) Pub. Date: May 6, 2004

(54) Method and device for characterising and/or for detecting a bonding complex

(76) Inventors: Hermann Gaub, Neuhaus (DE); Christian Albrecht, Munchen (DE); Filipp Oesterhelt, Wuppertal (DE)

Correspondence Address: NEEDLE & ROSENBERG, P.C., SUITE 1000, 999 PEACHTREE STREET, ATLANTA, GA, 30309-3915, US

(21) Appl. No.: 10/344,212

(22) PCT Filed: Aug. 9, 2001
(86) PCT No.: PCT/EP01/09206

(30) Foreign Application Priority Data
Aug. 11, 2000 (DE) 100 39 393.4
Oct. 16, 2000 (DE) 100 51 143.0
Apr. 10, 2001 (DE) 101 17 866.2

(51) Int. Cl. C12Q 1/68

(52) U.S. Cl. 435/6; 436/518

(57) ABSTRACT

A method of characterizing and/or identifying a binding complex, comprises the steps:

preparing a first binding partner and a conjugate of a second and a third binding partner and preparing a fourth binding partner,

forming an interlinkage of the binding partners, wherein the first binding partner with the second binding partner forms a sample complex and the third binding partner with the fourth binding partner forms a reference complex,

applying a force to the interlinkage which results in unbinding of the sample complex or the reference complex, and

determining which of the two binding complexes was unbound.

The invention concerns a method and an apparatus for characterizing and/or identifying a binding complex, in particular by means of distinguishing molecular unbinding forces by a differential force test.

Binding Tests on the Basis of Equilibrium Constants

Non-covalent interactions between molecules are based on the atomic interaction of binding partners by hydrogen bridges, ionic, hydrophobic and van der Waals forces. Weak interactions are orders of magnitude less than covalent bonds which are formed or dissolved by chemical reactions.

Non-covalent interactions between binding partners with a high degree of selective binding property are the prerequisite for molecular detection which is put to use in chemical analysis and diagnostics. Hereinafter such interactions are referred to as specific interactions. The methods of identifying or characterizing biochemical modules generally involve binding tests.

A binding test is based on the formation of a binding complex by the specific interactions of a ligand with a receptor and the identification of that complex.

In a diagnostic binding test, the important consideration is to identify a known substance in a sample:

to determine the identity of a sample substance,

to identify a sample substance in a complex sample mixture,

to distinguish variants of a sample substance, and

to determine the concentration of a sample substance.

In binding tests for the development of new diagnostic agents or therapeutic agents, the important consideration is to find for a given binding partner a suitable, as yet unknown second binding partner, that is to say:

identify out of a plurality of sample substances that which binds to a given receptor,

determine the binding constant of the sample substance to the receptor, and

determine further binding properties such as the rate of association (on rate) or dissociation (off rate).

In immunodiagnostics the receptors are generally antibodies or antibody derivatives, with which it is possible to detect and identify antigens in the form of proteins, low-molecular substances but also viruses and whole cells.

In molecular diagnostics, in regard to the receptor reference is made to a probe which comprises a nucleic acid such as DNA or RNA and with which nucleic acids are identified in a sample.

The most wide-spread immunodiagnostic test is the enzyme linked immunosorbent assay (ELISA). In the ELISA procedure a first antibody is immobilized on a surface. It selectively binds an antigen of an added sample mixture by way of a first binding site (epitope). A second antibody which is provided with a marker and which is in free solution binds to a second epitope of the antigen. In a separation step the fraction of the marked antibody, which has not bonded to the antigen, is separated off. The sandwich complexes which remain behind on the surface, comprising the first antibody, the antigen and the second antibody are identified by means of the marker. An enzyme which develops the signal by means of forming a color can generally bond to the marker. The color is ultimately the measurement in respect of the amount of antigen in the sample being investigated.

A typical molecular-diagnostic test is Southern hybridization. It is based on the interaction of a known nucleic acid molecule, the probe, in relation to a complementary nucleic acid of a sample mixture. The sample is immobilized on the surface and the marked probe is added. Under suitable buffer and temperature conditions the probe molecules bind to complementary sequences of the sample. After separation of the non-bound probe the nucleic acid duplexes formed comprising probe and sample molecules are quantified by means of the marker.

In reverse Southern hybridization which is the basis for highly parallel nucleic acid analysis on miniaturized probe arrangements (DNA microarrays), the probe molecules are bound on the surface and marked sample molecules are added in free solution.

ELISA and Southern hybridization have the common aspect that the binding result is identified by a marking procedure and that one of the binding partners is bound on a surface. A further property which they have in common with all current binding tests is that binding properties of two binding partners in relation to each other are characterized on the basis of the magnitude of the binding energy in the resulting binding complex.

An analytical method in which a force component is also used is described by WO99/45142. This involves separation of a nucleic acid complex as soon as it is bound by the deposit of a sample substance with a tensile force. Separation of that complex results in a fluorescence signal by virtue of the spatial separation of two fluorophores.

In that respect there is no provision for the fact that, instead of the nucleic acid duplex, it would also be possible to separate the binding complex of the sample substance. There is therefore no force comparison between the binding forces of two binding complexes. On the contrary, WO99/45142 can only provide for determining the presence and absence of an analyte.

It was not realized in WO99/45142 that the magnitude of the binding force between the sample substance and the receptor represents an item of valuable analytical information, nor was a means for determining same described.

Model of Molecular Interaction

The chemical interaction between two binding partners can be described by means of various models. The classic theoretical structure is thermodynamics. For the development of thermodynamics the decisive factor was that for a long time it was not possible to measure molecular interactions at individual molecules. Therefore it describes the interaction of particles on the basis of macroscopically measurable parameters. That results in the concept of binding energy which is defined as the energy required to dissolve a bond. The binding energy of two binding partners relative to each other can be deduced by way of measurement of the levels of concentration of free and bound binding partners by way of the equilibrium constant.

A basically different concept for the characterization of molecular interaction was made possible by force measurement, for example with an atomic force microscope, at individual molecules. The forces which are produced between the atoms of two binding partners can be experimentally determined by the unbinding force to be applied. Under certain conditions the binding potential of a binding complex can be reconstructed from the rate dependency of the unbinding forces. In contrast to characterization of a binding complex on the basis of its binding energies, decomposition of the complex upon force measurement is governed not by thermal excitation but by a manipulative intervention.

A greatly simplified binding model is described here to explain the binding potential.

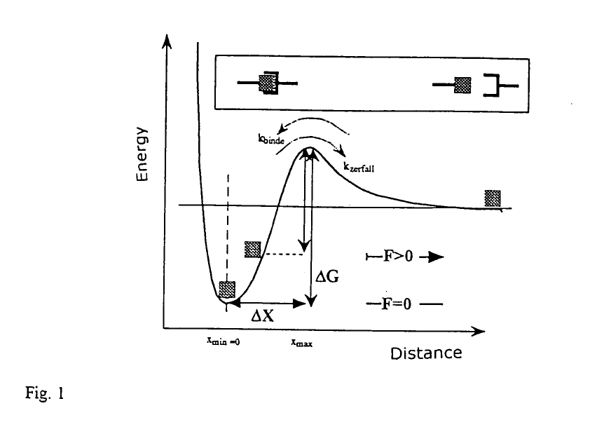

A binding complex is not characterized solely by its binding energy. It also has an activation barrier which is characteristic of it and which determines its binding and decomposition probability. Each binding complex also has a defined spatial structure. A characteristic parameter describing that structure is the binding length or potential width. Those parameters can be summarized in the following simple model of the binding potential which is shown in FIG. 1.

In the bound condition the ligand is at the minimum of the potential. In order to break up the complex the ligand must be pulled over the potential barrier out of the binding pocket or out of the binding potential. The force required for that purpose is determined by the derivative of the potential (gradient).

Superimposed on the mechanical breaking action is spontaneous decomposition of the complex by thermal activation. That spontaneous decomposition is dependent on the activation barrier ΔG. A force applied to the complex lowers the activation barrier which is still to be overcome. With any applied force the complex thus has a certain probability of decomposing within a given time. The force at which a complex actually decomposes therefore also depends on how quickly the force is applied. If the applied force is increased continuously at a fixed force rate r until the complex decomposes, that gives the following mean unbinding force for the complex, in dependence on the applied force rate:

$$F = \frac{kT}{Δ x} \cdot 1 n [ \frac{Δ x \cdot r}{{k}_{decomp .} \cdot kT} ]$$

If at various force rates the respective mean unbinding force is determined, it is possible to determine therefrom the binding width Δx which is characteristic of the binding complex and the decomposition rate k_{decomp.} of the complex.

The measurement of unbinding forces therefore represents a new approach to the characterization of binding complexes.

Methods of molecular force measurement

Although the great majority of binding tests detect selective interactions on the basis of binding energies, there are examples of methods which enjoy the advantages of the characteristic unbinding forces of the binding complexes.

The first example of such an apparatus is the “surface force apparatus” (SFA) which was developed in 1976 by Israelachvili (U.S. Pat. No. 5,861,954, 1999, Israelachvili). That apparatus can be used to measure adhesion forces of two molecule layers relative to each other. For that purpose each of the sample substances is applied to a cylindrically curved mica sheet, which in the ideal case are brought into contact at only one point. By virtue of precisely controlled movement of the mica surfaces relative to each other, forces are applied between the molecular layers. If the movement of the surfaces relative to each other in dependence on the applied force is measured, that affords information about the adhesion forces between the two molecular layers.

The second method of measuring molecular forces involves the “atomic force microscope” (AFM). The AFM was the first to succeed in determining the unbinding force of a weak interaction of a biological receptor-ligand pair, the biotin-streptavidin system (E.-L. Florin, V. T. Moy and H. E. Gaub, Science 264, 415 (1994)). In contrast to the SFA the AFM does not involve macroscopic surfaces being brought into contact. The tip of an AFM is only a few nanometers in size and at its end in the ideal case can come to a point on an individual atom. In the best case scenario an individual molecule, for example a DNA double strand, can be suspended between an AFM tip and a second surface. If now the tip is pulled away from the surface, that causes stressing of the molecule and bending of the AFM cantilever, which, with a known spring constant of the cantilever, permits measurement of the molecular binding forces.

A modification of the AFM method which is aimed in the direction of diagnostic application is described in U.S. Pat. No. 5,992,226 to Lee et al. Here, instead of being on a flat surface, the sample is bound between a projection which terminates in a point and the AFM tip.

A third method of characterizing molecular forces is based on the use of microscopically small magnetic beads. A receptor is bound on a magnetic bead and same is caused to form a binding complex with a ligand which in turn is bound to a surface. If the magnetic bead is exposed to a defined magnetic field, a defined pulling force can be applied to the binding complex between the surface and the bead. If the position of the magnetic bead is observed in the direction of the pulling force while same is varied until separation of the binding complex occurs, it is possible to determine the unbinding force which is required to tear the receptor and the ligand apart.

The identification of unbinding forces with magnetic beads is derived from an immunological sandwich test in which the forces are used only for the separation of a bound and a non-bound ligand (U.S. Pat. Nos. 5,445,970 and 5,445,971, 1995, to Rohr). A more extensive approach provides for fine matching of the pulling forces so that in principle weak specific bonds of an antigen to an antibody can be distinguished from strong ones (patent WO9936577, 1999, to Lee).

The fourth method involves force measurement with “optical tweezers”. The bases of that procedure go back to Arthur Ashkin. Application of the pulling force is here implemented by the movement of a strongly focused laser beam which can capture and move a microscopic particle.

An application of the optical tweezers for force measurement for diagnostic purposes was described by Kishore (U.S. Pat. No. 5,620,857, 1997, Kishore et al).

The fifth method is based on the adhesion of an elastic pillar (conformal pillar) which is coated with a sample substance to a surface which is coated with a probe. When the pillar is detached from the surface again it is possible to measure unbinding forces which serve to identify the sample (patent EP 0 962 759, 1999, to Delamarche et al). Characterizing binding complexes on the basis of unbinding forces instead of on the basis of binding energies affords some significant advantages. By way of the potential width of the bond it is possible to obtain a new independent parameter for characterization of the bond, which makes it possible to distinguish from each other various binding modes which involve identical binding energies. That applies in particular in regard to distinguishing non-specific and specific bonds in relation to proteins, and for discriminating between nucleic acid duplexes which are fully complementary and mis-pairings.

An object of the present invention is to provide a method and an apparatus which permits a simple test.

That object is attained by the features of the claims.

A further aim of the present invention is to make the described advantages of binding tests which are based on distinguishing unbinding forces accessible for a wide range of applications and commercial use, which was not possible with the state of the art hitherto.

The invention has advantages over conventional force discrimination methods. The conventional methods of molecular force measurement are further developments of methods which originally served a completely different purpose. The SFA method was developed for surface forces, AFM for imaging surfaces, magnetic beads for the separation of molecules and the conformal pillar method was developed as a preparative method for the structuring of surfaces.

A further aim of the present invention is to provide a force test which can test binding properties of a binding complex by simultaneous testing of a plurality of non-cooperative individual events.

In the case of methods with magnetic beads, the result obtained is generally unbinding forces which are based on a plurality of cooperative events as a plurality of binding complexes are fixed to a bead.

A further aim of the present invention is a force test in which separation of the binding complex and detection of the result are separated in respect of time (one-off). That results in a simple apparatus structure and a high degree of flexibility in terms of selecting the detection method.

A further aim of the present invention is a force test which does not require a complex apparatus structure and the implementation of which does not require experts.

A further aim of the present invention is a force test which permits high parallel measurement of many different samples.

A further aim of the present invention is a force test in which tensile forces can be implemented, which are far above that of the optical tweezers and that of the magnetic beads.

A further aim of the present invention is that of providing faster binding kinetics in respect of the binding partners than are possible in a method such as ELISA, in which the kinetics are limited by the diffusion speed of the reaction partners which are in free solution.

In contrast to the described methods the present invention is based on a procedure which was designed from the outset for determining binding potentials and which affords significant advantages over the state of the art.

The surface force apparatus by Israelachvili is not suitable for the analysis of biomolecules. The apparatus structure is extremely complex and costly and for that reason is scarcely suitable for diagnostic purposes. Parallel measurement of various substances is not described and it is thought it could only be implemented with difficulty.

The main advantage of the atomic force microscope is a high level of force resolution. The complex apparatus structure however results in high procurement costs and also handling which requires an expert makes that piece of equipment unsuitable for use outside basic research. A further limitation is that a statistically guaranteed measurement result requires a plurality of sequential experiments and therefore involves a large amount of time. AFM also suffers from an inherent disadvantage in regard to one of the most important demands in terms of a diagnostic measurement method, the parallel measurement of many different sample substances.

The use of magnetic beads gives rise to problems in regard to the application of forces. Practicable particle sizes and field strengths do not permit forces which would be capable of pulling a DNA-duplex apart, which excludes that method from use in molecular diagnostics. The same problem applies in regard to the use of optical tweezers. Here the pulling forces which can be produced are markedly below 100 pN and are therefore much too low to be able to test a DNA-duplex.

The present invention can have the following advantageous properties:

simple and advantageous apparatus structure

simple handling

a high level of force resolution with the simultaneous measurement of a plurality of binding events

unlimited pulling force

non-cooperative simultaneous testing of many binding complexes

The possibility of simultaneously testing many similar sample complexes during a measuring procedure, wherein the result for each complex occurs independently of the other complexes, that is to say it is non-cooperative, is to be viewed as one of the main advantages of the invention. The described methods with the conformal pillar or with magnetic beads always involve cooperative events as separation of some of the complexes has an effect on the separation probability of the remaining complexes. In that case the information about the individual events is lost.

The terms used to describe the invention are defined as follows:

Suspension: connecting a binding partner to a holding device.

Binding properties: relationship of two binding partners with each other such as: no binding; binding affinity; binding mode.

Binding complex: a complex comprising a plurality of binding partners; molecules or bodies or bodies and molecules which are in interaction relationship with each other and which can be separated by a pulling force.

Binding partner: constituent of a binding complex which can be separated by pulling forces from another binding partner. Binding partners can interact with each other specifically or non-specifically. The interaction is non-covalent.

Biomolecules: molecules which are obtained from biological systems or artificial molecules which are the same as those from biological systems.

Conjugate: connection of two binding partners.

Connection: connecting element of a conjugate.

Reference complex: binding complex with a unbinding force as a reference value or a known unbinding force.

Ligand: one of the binding partners of a specific binding complex.

Mean unbinding force: arithmetic mean of the unbinding forces of a plurality of similar binding complexes whose individual unbinding force varies by virtue of thermal excitation.

Sample (or target): molecule, polymer etc. which can form a sample or target complex.

Sample or target complex: binding complex which is to be characterized/identified. Either this involves two known binding partners whose binding properties are to be determined or this involves a known binding partner. This can involve an unknown or a known unbinding force.

Receptor: one of the binding partners of a specific binding complex.

Separation: binding partners which do not involve a binding complex separate from those which involve a binding complex.

Specific interaction: molecular interaction between two binding partners of a binding complex, which is distinguished by a high degree of molecular recognition.

Unbinding force (or separation force): maximum force required for mechanically separating a binding complex.

Interlinkage: arrangement of a first binding partner which binds to a second binding partner of a conjugate and a third binding partner which is a constituent of the conjugate and which binds a fourth binding partner.

Coupling: connecting the two holding means by way of a interlinkage.

Coupling partner: two elements which bind to each other and in that way implement coupling.

Holding means: means by way of which a force can be applied to the interlinkage.

Coupling number: number of couplings actually effected in a test run.

Coupling efficiency: the quotient of the number of couplings actually made (coupling number) and the number of maximum possible couplings:

$$coupling efficiency = \frac{coupling number}{maximum possible couplings}$$

The invention is described in greater detail hereinafter by means of Examples and the drawings in which:

FIG. 1 shows a simplified binding potential of a binding complex,

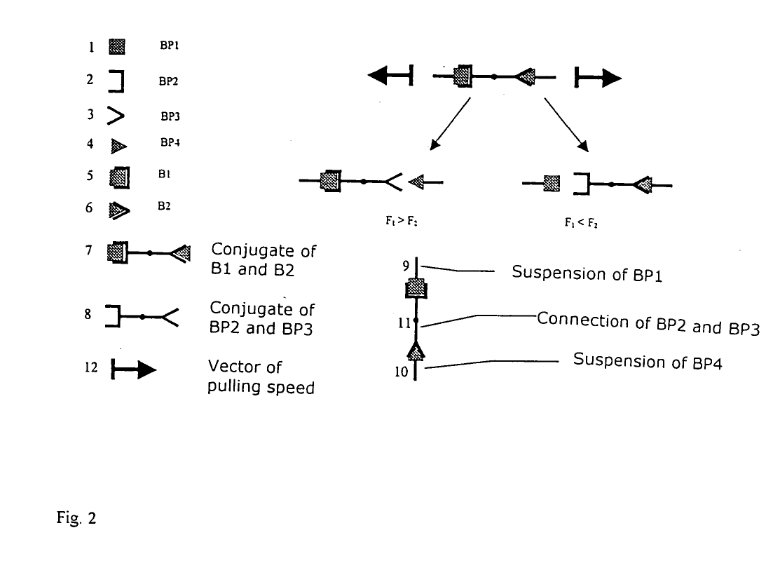

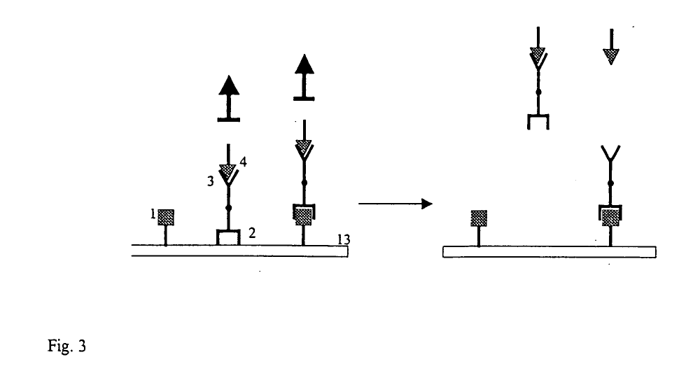

FIG. 2 shows the principle of the differential force test; after the application of a pulling force to the conjugate of B1 and B2 tearing of B1 occurs if F₁<F₂ or tearing of B2 occurs if F₁>F₂,

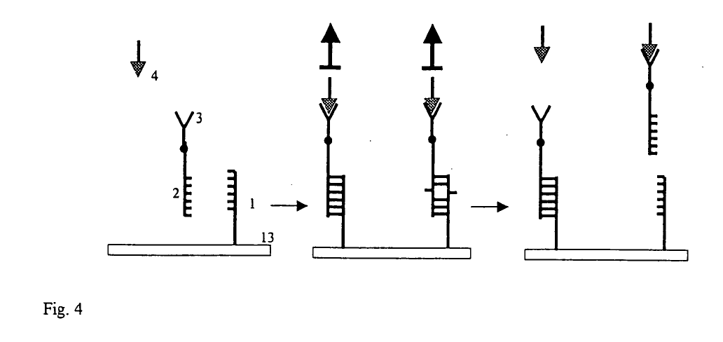

FIG. 3 shows the distinction between non-specific interactions with a body and specific interactions with a binding partner,

FIG. 4 shows the distinction of a completely paired nucleic acid duplex from an incompletely paired one,

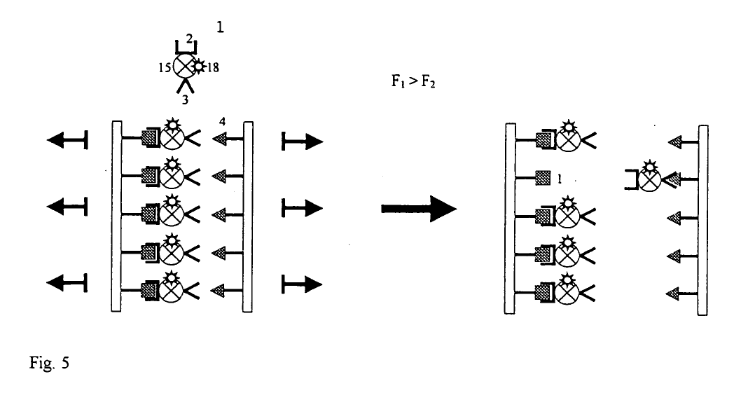

FIG. 5 shows the simultaneous implementation of a comparative force test on five independent similar binding complexes in a sandwich format; in this case the sample has the binding partners BP2 and BP3, the sample is provided with a marking; as F₁>F₂ the binding complexes B2 predominantly tear, and

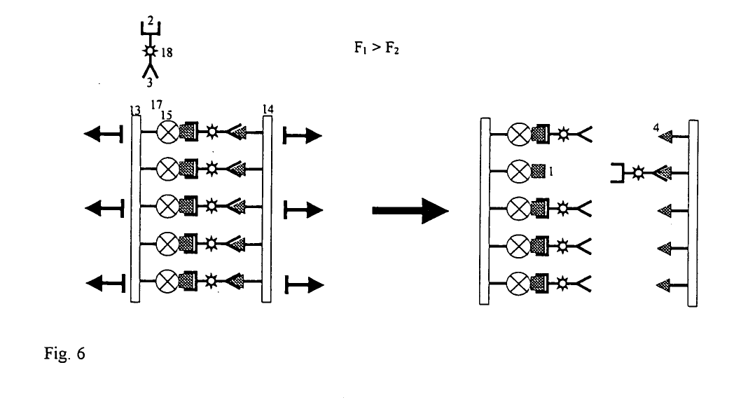

FIG. 6 shows the simultaneous implementation of a comparative force test on five independent similar binding complexes in a capture format; in this case the sample has the binding partner BP1 and is bound to the surface 1, the conjugate of BP2 and BP3 is provided with a marker; as F₁>F₂ the binding complexes B2 predominantly tear,

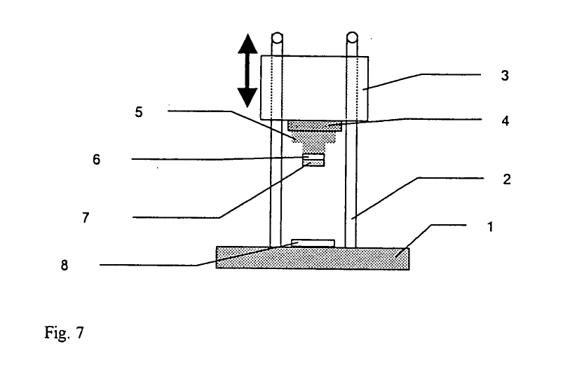

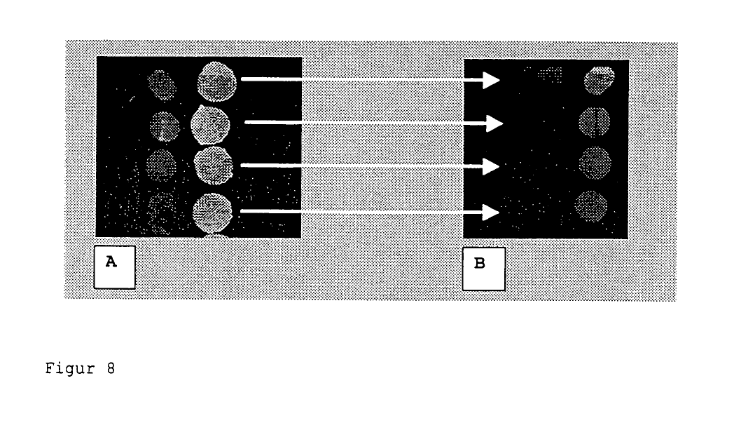

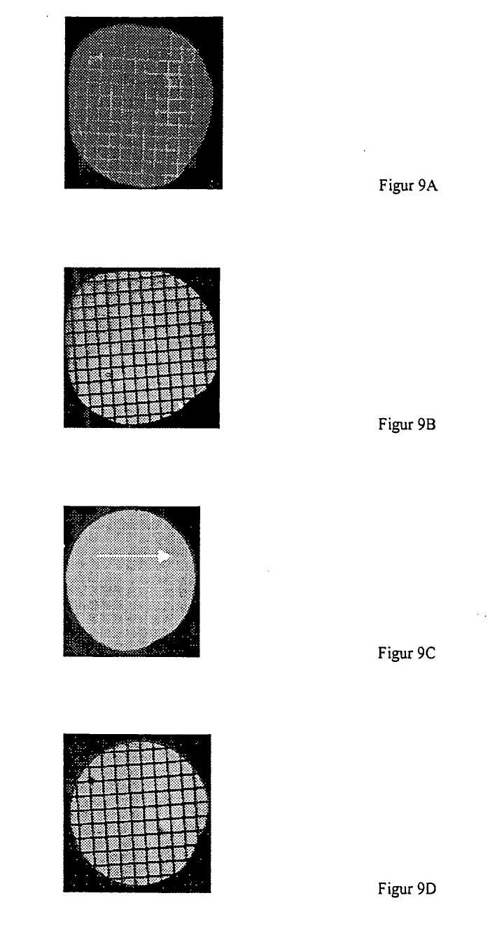

FIG. 7 shows a possible embodiment of a pillar apparatus which is suitable for carrying out the method according to the invention, a more precise description of a possible construction is to be found in Experimental Example 1,

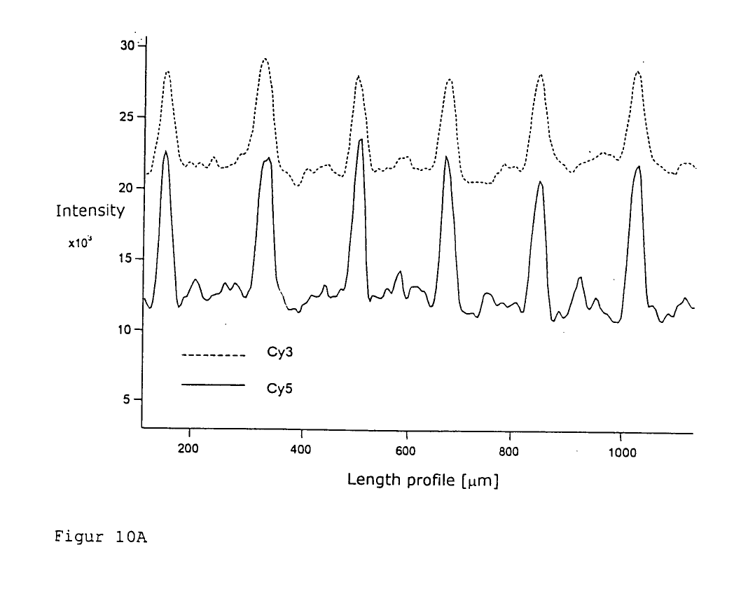

FIG. 8 shows views of a support or substrate (8A) and a pillar (8B) after a force test for comparison of the complexes biotin/streptavidin and iminobiotin/streptavidin (see Experimental Example 1),

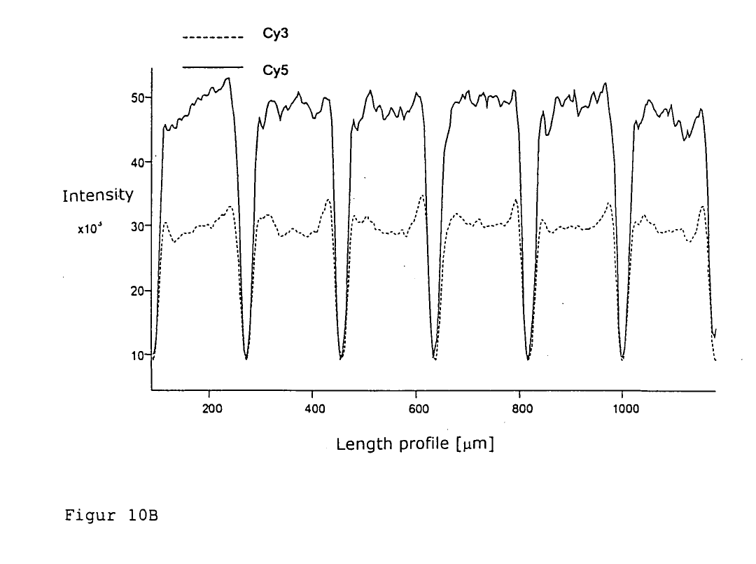

FIG. 9 shows respective views of a substrate (9A and 9C) and a pillar (9B and 9D) after force tests for the comparison of two DNA-duplexes (see Experimental Example 2), FIG. 9A showing the substrate in Experiment 2a, FIG. 9B showing the pillar in Experiment 2a, FIG. 9C showing the substrate in Experiment 2b and FIG. 9D showing the pillar in Experiment 2b,

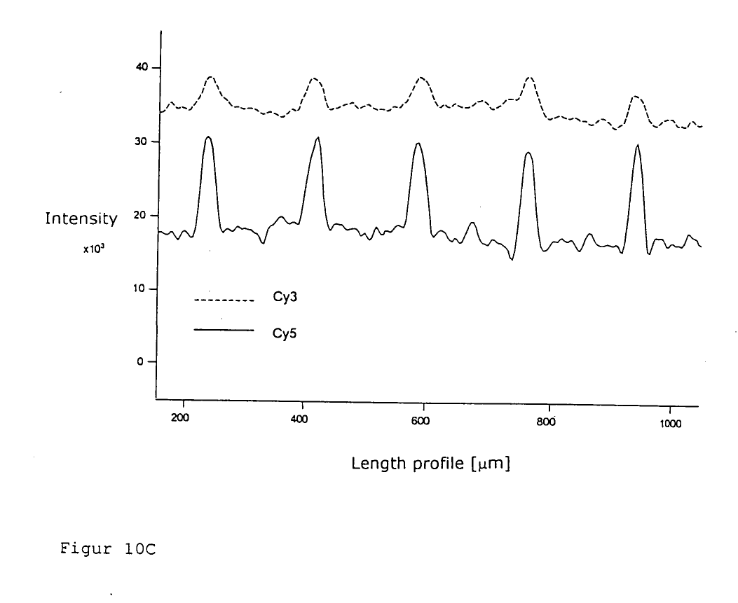

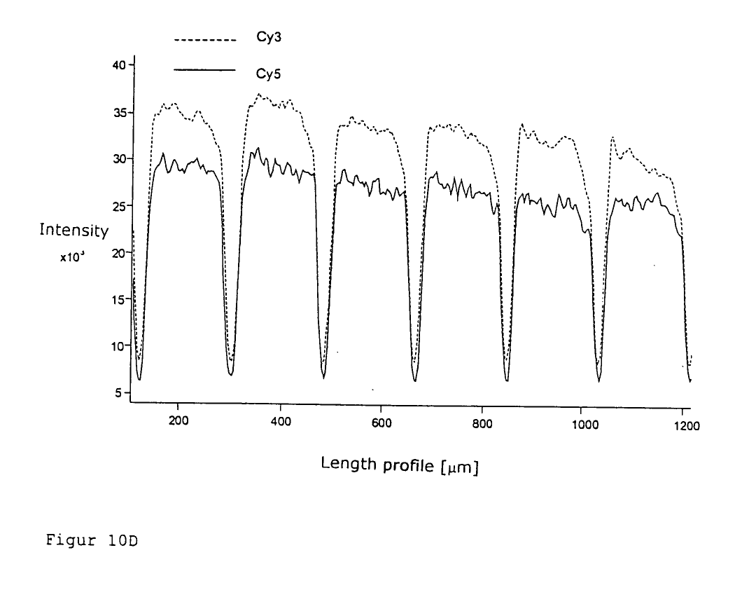

FIG. 10 shows the results of evaluation of the substrate and the pillar after a force comparison, wherein a DNA-duplex was compared to an identical duplex (10C and D) and a further duplex (10A and B) (see Experimental Example 2), 10A: substrate in Experiment 2a; 10B: pillar in Experiment 2a; 10C: substrate in Experiment 2b; 10D: pillar in Experiment 2b; these respectively involve portions of fluorescence profiles,

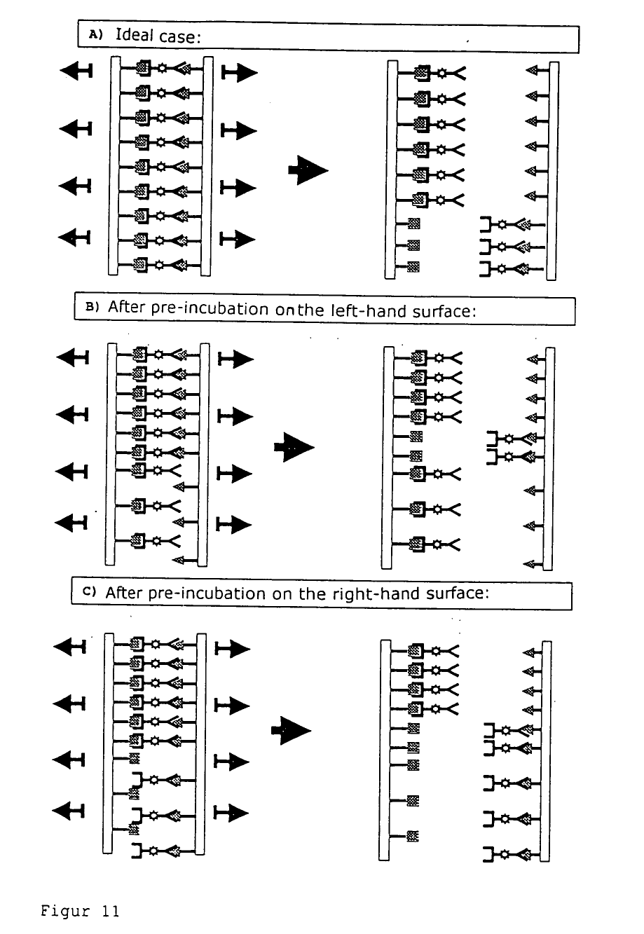

FIG. 11 diagrammatically shows the distribution of the conjugate with complete coupling (A) and partial coupling (B and C),

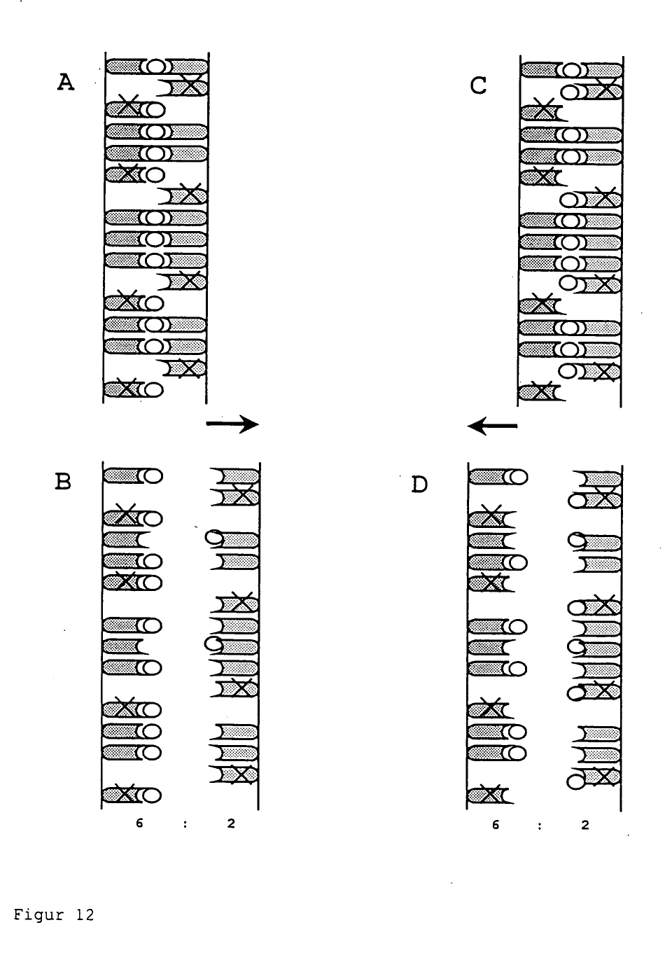

FIG. 12 shows the principle of the reverse pillaring procedure, A and C respectively showing the surfaces which are brought together during the pillaring operation, with the marked sample being bound on the one hand on the left-hand side (A) and on the other hand on the right-hand side (B), wherein the binding partners identified by “X”, with a level of coupling efficiency of 2/3, do not form a bond to the respective other surface, and they are therefore also not involved in the distribution between the surfaces in the separation operation,

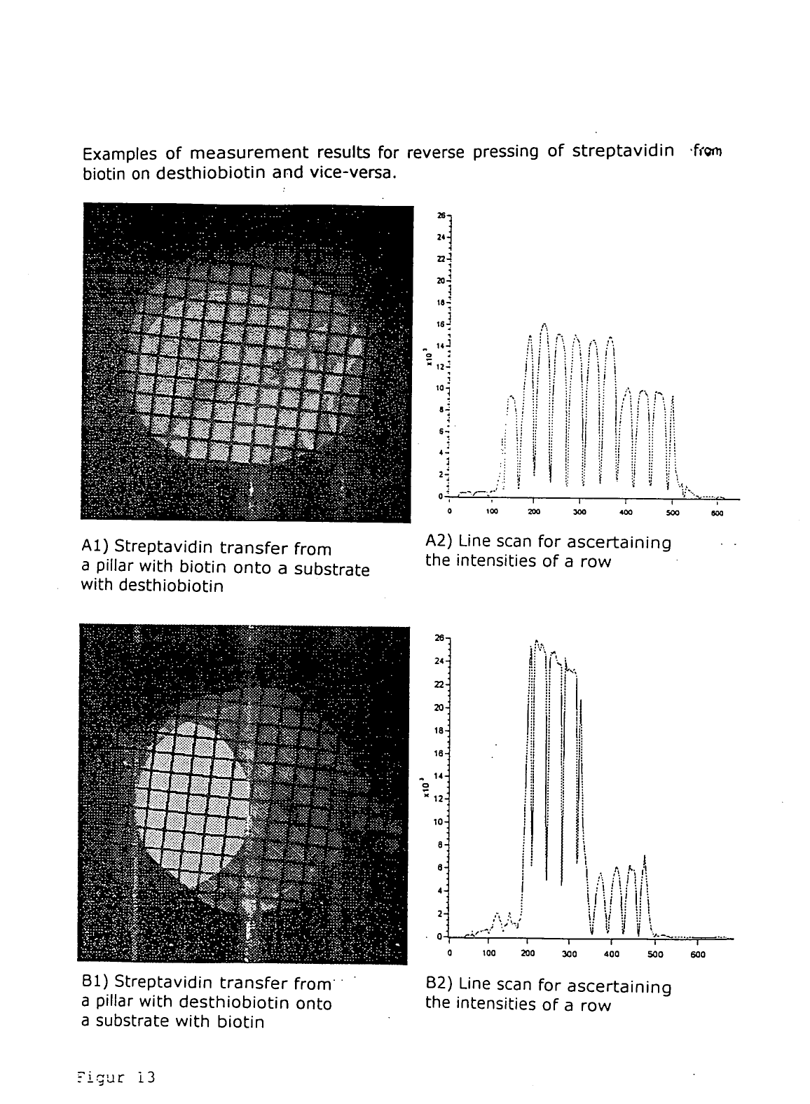

FIG. 13 shows examples of measurement results for reverse pillaring of streptavidin from biotin on desthiobiotin and vice-versa, and

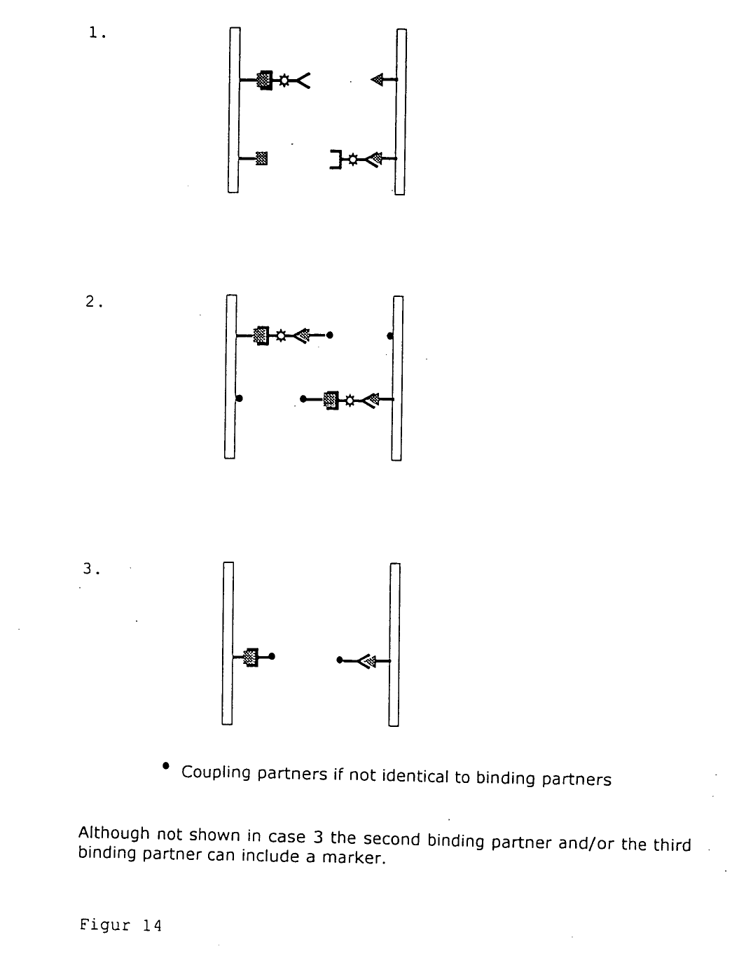

FIG. 14 shows various ways in which coupling can be effected.

1. First binding partner BP1

2. Second binding partner BP2

3. Third binding partner BP3

4. Fourth binding partner BP4

5. Binding complex 1 (B1)

6. Binding complex 2 (B2)

7. Interlinkage

8. Conjugate of the second binding partner BP2 and the third binding partner BP3

9. Suspension of the first binding partner BP1

10. Suspension of the fourth binding partner BP4

11. Connection of the second binding partner BP2 and the third binding partner BP3

12. Vector of the pulling speed

13. Surface 1

14. Surface 2

15. Sample

16. Sample with the second binding partner BP2 and the third binding partner BP3

17. Suspension of the sample

18. Marking

The present invention involves a method and an apparatus for carrying out that method, which in comparison with the conventional force tests involve a completely different principle for determining unbinding forces.

A differential force test comprises two binding complexes which are linked together. Upon the application of a force which is at least above the unbinding force of one of the two binding complexes, tearing of one of the two binding complexes takes place. The binding complex with the higher unbinding force remains intact in that situation. If the unbinding force of one of the two binding complexes is known, it is possible in that way to conclude whether the unbinding force of the second binding complex is higher or lower than that of the first one. The differential force test can be used for a large number of diagnostic applications. The invention is suitable in particular as a method of diagnostic detection and identification or for characterizing the binding properties of biochemical molecules or molecules with a high degree of specific molecular recognition.

The present invention provides that characterization of binding properties of binding partners is effected on the basis of the unbinding force which is necessary for separation of the binding complex thereof.

The differential force test according to the invention can be implemented on a single interlinkage. It is preferred however that a plurality of similar interlinkages are used in a force test. If in the present application certain constituents of the interlinkage and/or method steps are referred in the singular (for example binding partner, binding complex, conjugate, interlinkage, coupling partner, sample or target and so forth), that does not mean that the invention is limited to force tests on individual interlinkages. On the contrary that also embraces force tests with a plurality of interlinkages. The use of the singular only serves to make the discussion of the invention easier to follow. It is known to the man skilled in the art that in practice a test is generally carried out on many molecules, binding partners, complexes and so forth.

The procedure for characterizing the unbinding force F₁ which has to be applied for the separation of a binding complex B1 (5) is effected by comparison with the reference unbinding force F₂ which has to be applied for the separation of a second binding complex B2 (6). Both binding complexes are connected to form a interlinkage, to the two sides of which a pulling force is applied. Reference is made to coupling when a interlinkage connects two holding means, by way of which a force can be applied. It is to be emphasized that the pulling force applied to the two serially arranged binding complexes is equal in magnitude. If the pulling force exceeds a value which is above one of the unbinding forces (F₁ or F₂), that results in separation of the binding partners of that binding complex whose unbinding force is lower. If for example separation of the binding complex B1 occurs, it follows therefrom that F₁<F₂. If B2 were separated, it follows that F₁>F₂. FIG. 2 shows the principle of the force test.

The binding complex B1 (5) generally involves a sample complex, that is to say a binding complex whose binding properties are to be characterized or in which a binding partner is to be identified by way of a known binding property, with another binding partner. This however may also involve an undefined non-specific interaction between two binding partners or between a binding partner and a body.

B2 (6) generally involves a reference complex, that is to say a binding complex whose binding properties predetermine a parameter, in particular a unbinding force, to which the sample complex is compared.

Usually the sample complex includes the first binding partner BP1 and the second binding partner BP2, while the reference complex includes the third binding partner BP3 and the fourth binding partner BP4. In accordance with the present invention however the sample complex may also include the third binding partner BP3 and the fourth binding partner BP4 and the reference complex may include the first binding partner BP1 and the second binding partner BP2.

The principle as described hereinbefore does not involve “measurement” in the narrower sense but rather “gauging”. In accordance with the German Industrial Standard (DIN) the term “gauging” describes a testing procedure in which the item to be tested is compared to a known value or magnitude of another item. In that sense, the present invention also provides that the unbinding force of the binding complex B1 is compared to a second known unbinding force. “Measuring” in contrast involves a testing procedure in which the value to be determined gives a specific numerical value on the measuring scale. That corresponds to the situation of force measurement with the AFM or with one of the other methods in the state of the art.

The particularity of the present invention involves associating with each sample complex to be tested a force gauge on a nanoscopic scale, being the reference complex. Each sample complex is tested independently, the result of many individual tests finally gives the measurement result. A particularity which follows from that principle is the possibility of being able to implement the separation of the binding complexes and the detection operation, separately in respect of time.

The invention takes account in particular of thermal excitation. It will be apparent from the model discussed hereinbefore of molecular interaction (FIG. 1) that the unbinding force which is required to separate the binding complex B1 or a further binding complex B2 varies as the interaction between the binding partners is subjected to thermal excitation. Therefore in accordance with the present invention, the comparison of the two binding pairs is preferably effected a plurality of times in order to be able to form a statistically secure mean value, the mean unbinding force, which says whether F₁>F₂ or F₁<F₂. That occurs by exposing as many similar binding complexes to the same unbinding force at the same time and determining how many of the binding complexes B1 and how many of the binding complexes B2 were separated.

The invention additionally or alternatively takes account of the force rate dependency. An important parameter in terms of implementing a differential force test is the rate of the applied pulling force as the unbinding forces F₁ and F₂ can vary greatly, at different force rates. In order to be able to reproducibly repeat a differential force test in accordance with the above-described principle, it can be crucial to operate with only one given force rate.

The force rate is determined by the speed of the pulling force and the elasticity of the conjugate of the two binding complexes together with the suspension of the first binding partner BP1 and the fourth binding partner BP4.

Depending on the respective application of the differential force test, it is also possible to deliberately vary the force rate in order to obtain a given piece of information about a binding complex.

In a preferred embodiment the invention takes account of the number of couplings or the efficiency with which a interlinkage comprising the binding partners BP1, BP2, BP3 and BP4 is coupled between the two holding means. Particularly when comparing two unbinding forces of similar magnitude, it is advantageous to include the coupling number and/or the coupling efficiency in evaluation of the test, in order to be able to ascertain the actual ratio of the unbinding forces.

Examples of use of the invention:

1. Distinguishing binding modes

1.1. Distinguishing specific and non-specific interactions

A central problem of binding tests in which a first binding partner is immobilized on a surface is the non-specific background signal. The non-specific background signal is caused by molecules of a second binding partner which were added in free solution and which have non-specifically bound to the surface. That therefore involves superimposition in respect of the specific signal, that is to say the signal of those molecules of the second binding partner, which are specifically bound to the first binding partner. As non-specific and specific interactions can bind with binding energies of similar magnitude, it is difficult to distinguish them by a binding test which is based on discriminating between binding energies.

FIG. 3 shows a differential force test for distinguishing non-specific and specific binding. The conjugate of BP2 and BP3 binds non-specifically to the surface and specifically to the binding partner BP1 which is immobilized on the surface and with which it forms the binding complex B1. BP4 forms with BP3 a binding complex B2. For a given force rate the unbinding force F₂ of the binding pair B2 is higher than the unbinding force of the non-specific binding of the binding partner BP2 with respect to the surface. The specific unbinding force F₁ of BP2 and BP1 is however higher than F₂. After the pulling force is applied therefore separation of the non-specifically bound conjugate occurs, but not of the specifically bound one.

1.2. Distinguishing weaker specific bonds from stronger specific bonds in the example: distinguishing an individual base mis-pairing in a nucleic acid duplex from a complete pairing.

A serious problem in terms of distinguishing nucleic acid sequence variants by reverse Southern hybridization is that of distinguishing nucleic acid sample molecules which are bound to the immobilized probe, to ascertain whether they are completely complementarily bound or whether they have an individual base mis-pairing. In a test with only one given probe of about 15-25 base pairs individual base mis-pairings can also be distinguished from complete pairings on the basis of the binding energies. For that purpose the hybridization procedure is carried out near the melting temperature of the completely paired complex. Under those conditions the mis-pairing is unstable. In the case of an arrangement of a plurality of probes of various sequences, as is generally the case with reverse hybridization however, that option does not apply.

FIG. 4 shows distinguishing a complete base pairing from an individual base mis-pairing.

The invention is carried into effect with the following means:

1. Exerting Forces

The operation of exerting pulling forces to the interlinkage can be implemented in very different ways, in which respect the nature of the applied force is not an important consideration.

In the preferred embodiment the pulling force is a mechanical macroscopic tension. For that purpose the interlinkage is fixed between two bodies and they are moved away from each other until separation of one of the two complexes occurs. The bodies can be nanoscopically small, but this may also involve macroscopically large surfaces.

In the second case the pulling forces are produced by magnetic particles which are fixed to the interlinkage and on which a magnetic field acts. This may involve two different particles which are each fixed to a respective end of the interlinkage and which in one case have diamagnetic properties and in the other case paramagnetic properties.

A third case makes use of the possibility of connecting the interlinkage to large molecules or polymers and producing pulling forces by means of the resistance thereof in a flow of fluid. Dynamic pulling forces can be built up if the interlinkage is bound between particles into which sound waves and in particular ultrasound can be coupled.

A fourth case involves making use of the influence of an electrical field on charged molecules, as is the case for example with an electrophoretic method. For that purpose the interlinkage is connected at least at one end to a charged molecule, preferably a multiply charged polymer. When both ends are joined to a charged molecule, these involve oppositely charged molecules.

In a fifth case the force is applied by shortening a polymer which forms the suspension means of the binding partners BP1 and BP2 or the connection between BP2 and BP3. In that case the shortening effect is based on a confirmational change in the polymer which is caused by a variation in the chemical medium, for example the pH-value or a salt concentration.

2. Variation in the Force Rate

The force rate with which a tension is applied to a molecule is determined by two parameters. On the one hand it is the spring constant of the suspension means of the binding partners BP1 and BP4 or the spring constant of the connection between BP2 and BP3, and on the other hand it is the pulling speed. In order to vary the force rate either a different spring constant or a different pulling speed is adopted.

The preferred mode for varying the spring constant of a suspension or a conjugate involves varying the length of a polymer which forms the suspension or the connection.

3. Detection

The purpose of detection is to determine which of the two binding complexes of a interlinkage was separated after the application of a pulling force. That can be done indirectly or directly.

Indirect detection is directed to a free binding partner BP which, prior to the application of the pulling force, was part of a binding complex B. That is achieved by adding a probe which is directed towards the free binding partner. If for example separation of the binding complex B1 occurs, it is possible to detect BP1 and/or BP2.

Direct detection involves detecting in which of the two pulling directions the conjugate of the binding partners BP3 and BP2 was displaced after tearing occurred. For that purpose the conjugate comprising BP3 and BP2 is provided with a marker. The operation of determining which of the two complexes was separated can be effected by determining the amount of conjugate of BP2 and BP3 which, after application of the force and after separation, is on one of the surfaces or holding means. The determining operation can also be effected by determining the amount of conjugate of BP2 and BP3, which after application of the force and after separation, is on the first holding means, and determining the amount of conjugate of BP2 and BP3 which, after application of the force and after separation, is on the second holding means.

A very wide range of different methods in the state of the art can be put to use for detecting and identifying the probe in indirect detection or the marking in direct detection. The preferred embodiment involves marking around a fluorescing molecule. Here it is also possible to use FRET (Fluorescence Resonance Energy Transfer), by a procedure whereby the two binding partners of a binding complex are provided with two different fluorophores, between which resonance transfer takes place. The fluorescence signal is lost upon separation of the two fluorophores. A further modification involves providing one of the two binding partners of a binding complex with a fluorophore and the other with a molecule which quenches the fluorescence of the fluorophore. Upon separation of the binding partner quenching of the fluorophore is eliminated and therefore an increase in the strength of the signal occurs. Preferred fluorophores used are nano-scale, colloidal semiconductor particles (quantum dots). Further options are radioactive marking, marking with an affinity marker to which an enzyme which strengthens the signal binds, chemoluminescence, electrochemical markings or mass spectroscopy.

4. Samples

The differential force test described herein can be used for a wide range of samples. Preferably these involve proteins, generally antibodies, antigens, haptens or natural and synthetic nucleic acids. This however may also involve viruses, phages, cell constituents or whole cells. It is moreover also possible to consider complex-forming substances such as chelating agents.

5. Reference Complexes

A binding partner of a reference complex can itself be a constituent of a sample, as is the case with the sandwich format. A reference complex can in principle be made up from the same constituents as a sample complex.

6. Time Sequence of the Interlinkage

The connection of the binding partners PB2 (2) and BP3 (3) to form a conjugate (8) can occur before the interaction of BP2 and BP3 with BP1 or BP4 takes place. Alternatively the conjugate of BP2 and BP3 can also be formed only after an interaction with BP1 and BP4 has occurred.

7. Coupling

The term coupling is used to indicate the connection of a interlinkage to the two holding means. The two elements involved in the coupling are referred as the coupling partners (KP1 and KP2). The coupling partners may involve binding partners, as is the case with case 1 (see below). In other cases however the coupling partners are different from the binding partners (see cases 2 and 3). Coupling will be discussed here by way of the example of a preferred embodiment. When using two surfaces as holding means, macroscopic tension for applying forces, and marking on binding partner 2 or 3 or on the conjugate, coupling can be effected in various ways; FIG. 14 diagrammatically illustrates the following cases:

1. BP1 is bound to the first surface. The conjugate of BP2 and BP3 is incubated on the first surface, in which case the complex B1 is formed from BP1 and BP2. The second surface is moved towards the first, this involving the formation of B2 from BP3 and BP4. The formation of B2 provides for both linking of the binding partners 1 through 4 and also coupling of the interlinkage to the two surfaces. BP3 and BP4 therefore also involve the coupling partners.

2. BP1 is bound to the first surface. The conjugate of BP2 and BP3 as well as BP4 are incubated on the first surface in such a way that the formation of a interlinkage takes place. The second surface is moved towards the first, this involving binding of BP4 to the second surface. This step involves coupling of the interlinkage already previously formed on the first surface. In this case BP4 is connected to a coupling partner which binds to a second coupling partner which is bound on the second surface.

3. BP1 is bound to the first surface. BP2 is incubated on the first surface, whereby the formation of B1 takes place. BP4 is bound on the second surface. BP3 is incubated on the second surface, whereby the formation of B2 takes place. The second surface is moved towards the first, this involving the formation of the conjugate of BP2 and BP3. This step involves both the formation of the interlinkage and also coupling of the interlinkage to the two surfaces. In this case BP2 and BP3 are each connected to a respective one of the coupling partners.

Ideally the ratio of the unbinding forces of B1 and B2 could be determined directly from the amount of conjugate which has remained on the first surface after implementation of the test and the amount of conjugate which was transferred onto the second surface. In practice however those values are falsified to a greater or lesser degree if it is not successfully possible to couple approximately all marked binding partners present. FIG. 11 clearly shows that.

In such a case the transfer of the marked conjugate onto the other surface depends both on the force ratio of B1 and B2, and also the amount of the coupled interlinkages (coupling number). A small transfer from for example the first to the second surface can indicate both that the unbinding force of B1 is greater than that of B2 and also that only a small number of interlinkages were coupled to the second surface. At the same time the amount of marked conjugates which have remained on the first surface is increased and thus falsified by those conjugates which were not coupled, that is to say not also subjected to force comparison.

If the number of couplings which actually took place (coupling number) is related to the maximum possible couplings, that gives the coupling efficiency. In that respect, the number of maximum possible couplings is limited by the number of that one of the two coupling partners which is in the smaller number. In accordance with the invention therefore it is further possible to at least approximately determine the coupling number or the coupling efficiency, namely the quotient of the number of the couplings actually formed and the number of maximum possible couplings, and possibly take account of the coupling number or coupling efficiency in ascertaining whether the sample complex or the reference complex was separated.

In order to exclude non-quantitative coupling as a source of error, there are various operating procedures:

a) reference experiment with a “self-comparison”

b) “reverse execution” of a test

c) “double marking” and pre-forming an interlinkage on a surface

The “self-comparison” (a) and “reverse execution” (b) will here be described in respect of case 1) (see above):

In a “self-comparison” (a), besides the actual force comparison, a reference experiment is also carried out. For force comparison, the marked conjugate is incubated with a first binding partner (for example BP1) which is bound on a first surface and the transfer to the second surface which is decorated with a further binding partner (for example BP4) is ascertained. In the force comparison procedure BP1 and BP4 involve different binding partners whose unbinding force ratio is to be ascertained. The reference test is carried out in the same manner, in which case the binding partners on both surfaces are identical to the binding partner BP4 (that is to say the binding partner of the force comparison on the second surface). In regard to the reference test, mention is made of a “self-comparison” as two identical complexes are compared.

In order now to be able to obtain information as to which of the binding complexes (B1 or B2) has the greater unbinding force, two prerequisites must be satisfied: identical coupling efficiency for force comparison and self-comparison and identical density of all binding partners which are bound to the first surface or all binding partners which are bound to the second surface respectively. Under those preconditions, it is possible to infer for B1 (complex of BP1 and BP2) a greater unbinding force if transfer onto the second surface turns out to be less in the case of the force comparison than in the case of the reference experiment. If in contrast the transfer is greater than in the reference experiment, then B2 (complex of BP3 and BP4) has the higher unbinding force.

Accordingly the invention also concerns a method in which (i) in a first step (=force comparison) the amount of conjugate including the second binding partner BP2 and the third binding partner BP3, which was transferred after the separation operation from a first holding means onto a second holding means is determined, and/or the amount of conjugate including the second binding partner BP2 and the third binding partner BP3, which after the separation operation was not transferred from the first holding means onto the second holding means is determined, wherein the first binding partner BP1 is different from the fourth binding partner BP4 and (ii) in a second step (=self-comparison), to determine the coupling efficiency, the amount of conjugate including the second binding partner BP2 and the third binding partner BP3, which after the separation operation was transferred from a first holding means onto a second holding means, is determined, and/or the amount of conjugate including the second binding partner BP2 and the third binding partner BP3, which after the separation operation was not transferred from the first holding means onto the second holding means, is determined, wherein the first binding partner BP1 is also used as the fourth binding partner BP4, or the fourth binding partner BP4 is also used as the first binding partner BP1, so that the first binding partner BP1 and the fourth binding partner BP4 in the second step are identical and the second binding partner BP2 is substantially identical to the third binding partner BP3.

A reference test with a self-comparison can however be carried out only if a conjugate with two identical binding partners is available, as is the case in Experimental Example 1. In cases of a different kind, it is necessary to have recourse to one of the following solutions.

In the “reverse execution” (b) of a test the marked conjugate is incubated in a first implementation with BP1 which is bound on the first surface and the transfer onto surface 2 is determined, with BP4. In a second implementation, the conjugate is incubated with BP4 on the second surface and the transfer onto the first surface is ascertained.

Accordingly the invention also concerns a method in which in a first implementation (i) the first binding partner BP1 and the conjugate including the second binding partner BP2 and the third binding partner BP3 are bound on a first holding means, the fourth binding partner BP4 is immobilized on a second holding means, the two holding means are moved towards each other so that the third binding partner BP3 and the fourth binding partner BP4 can bind to each other, the amount of conjugate including the second binding partner BP2 and the third binding partner BP3, which after the separation operation was transferred from the first holding means onto the second holding means, is determined, and/or the amount of conjugate including the second binding partner BP2 and the third binding partner BP3, which after the separation operation was not transferred from the first holding means onto the second holding means, is determined; and in a further implementation (ii) the fourth binding partner BP4 and the conjugate including the second binding partner BP2 and the third binding partner BP3 are bound on the second holding means, the first binding partner BP1 is immobilized on the first holding means, the two holding means are moved towards each other, so that the second binding partner BP2 and the first binding partner BP1 can bind to each other, the amount of conjugate including the second binding partner BP2 and the third binding partner BP3, which after the separation operation was transferred from the second holding means onto the first holding means, is determined, and/or the amount of conjugate including the second binding partner BP2 and the third binding partner BP3, which after the separation operation was not transferred from the second holding means onto the first holding means, is determined.

If the quotient is formed from the transfer (of conjugate) in the first and second (reverse) implementation, that also gives the ratio of the unbinding forces of the two binding complexes. As the coupling efficiency for both implementations can be assumed to be equal, it is shortened in the formation of the quotient thereof and therefore is no longer incorporated into the result. An embodiment of this procedure is described in Experimental Example 2.

The “double marking” (c) is discussed hereinafter in relation to case 2) (see above):

Case 2) involves using a second marking, the transfer of which onto the second surface corresponds to the coupling number. An embodiment of this procedure is described in Experimental Example 3. Unlike 1), in this case B1 and B2 are previously formed jointly on the first surface, in which respect the conjugate and BP4 are provided with distinguishable markings. Coupling occurs as soon as the coupling partner connected to BP4 binds to the second surface which carries the second coupling partner, by virtue of the two surfaces being moved towards each other. The amount of BP4 which has bound to the second surface thus corresponds to the coupling number. The procedure now involves determining the amount of the marked conjugate, which was transferred onto the second surface, or which was detached from the first one. If that amount is less than half the coupling number, it can be concluded that the complex of BP1 and BP2 has a greater unbinding force than the complex of BP3 and BP4. If the amount of conjugate transferred is greater than half the amount of the couplings, it can be concluded that the complex of BP1 and BP2 has a lower unbinding force than the complex of BP3 and BP4. An embodiment of this procedure is described in Experimental Example 3.

Preferably accordingly the conjugate of the second and third binding partners, the second binding partner or the third binding partner is provided with a first marker and the first or the fourth binding partner is provided with a second marker, the second marker being different from the first one.

Then, in an embodiment, the separation location is detected by ascertaining the amount of first marker which is bound to one of the holding means, ascertaining the amount of second marker which is bound to the same holding means, and comparing together and/or relating to each other the ascertained values. Information about the extent of the separation of sample or reference complex can be obtained from the ratio of the amounts of first and second markers, which are bound for example to the substrate or the pillar.

In an embodiment in that respect the interlinkage which includes the first, the second, the third and the fourth binding partners is firstly formed on a first holding means and then in a second step coupling to a second holding means is effected.

In another embodiment, either (i) the first binding partner BP1 and the conjugate including the second binding partner BP2 and the third binding partner BP3 are bound on a first holding means, the fourth binding partner BP4 is immobilized on a second holding means, the two holding means are moved towards each other so that the third binding partner BP3 and the fourth binding partner BP4 can bind to each other, or (ii) the fourth binding partner BP4 and the conjugate including the second binding partner BP2 and the third binding partner BP3 are bound on the second holding means, the first binding partner BP1 is immobilized on the first holding means, and the two holding means are moved towards each other so that the second binding partner BP2 and the first binding partner BP1 can bind to each other.

At least one of the binding partners may include a nucleic acid, in particular DNA. Preferably at least two of the binding partners include a natural or synthetic nucleic acid.

### PREFERRED EMBODIMENT OF THE INVENTION

In the first preferred embodiment of the invention, in which the conjugate of B1 and B2 is fixed between two surfaces, the pulling force is applied by a mechanical macroscopic tension and detection is effected directly by way of a marker.

This embodiment is discussed here in relation to the two formats which can also be implemented by all other embodiments, namely the sandwich format and the capture format:

In the sandwich format of the differential force test (FIG. 5) the conjugate of BP2 and BP3 involves the sample (15). A binding partner BP2 (2) of the sample is specific for a binding partner BP1 (1) which is bound on the first surface (13). A further binding partner BP3 (3) is specific for one of the binding partners BP4 (4), which is bound on the second surface (14). When the two surfaces are brought into contact interaction of the sample with BP1 and BP4 takes place, whereby the surfaces (13, 14) are joined by means of the binding complexes B1 and B2 produced. If now the surfaces are pulled away from each other, then preferably that one of the two binding complexes, which has the lower unbinding force, breaks. The sample adheres to that surface at which there is still an intact binding complex.

In the capture format of the differential force test (FIG. 6) the sample (15) is immobilized on the first surface (13). The sample has the first binding partner BP1 (1) which is specific for the second binding partner BP2 (2). BP2 is connected to BP3 (3) to form a conjugate, wherein BP3 binds a fourth binding partner BP4 which is bound on the second surface (14).

The two surfaces are brought into contact whereby interaction of BP1 with BP2 takes place. If now the surfaces are pulled apart, preferably the weaker of the two complexes breaks, that is to say either the complex of BP1 with BP2 or the complex of BP3 with BP4. The distribution of the conjugate of BP2 and BP3 between the two surfaces is determined and gives information as to which of the two complexes was the more stable.

Binding of the sample (15) in the capture format can be effected covalently or by way of weak interactions.

The preferred apparatus for carrying out the present invention comprises:

i) A use means which comprises two surfaces for binding of the reaction partners,

ii) The binding partners which are bound on the surfaces,

iii) An apparatus for bringing the two surfaces into contact and separating them again after molecular interaction of the binding partners has occurred,

iv) A marking means for the conjugate of the binding partners BP2 and BP3, on the basis of which the distribution between the two surfaces can be determined, and

v) An apparatus for detection of the marking means.

### EXPERIMENTAL EXAMPLE 1

Force test for comparison of the complexes biotin/streptavidin and iminobiotin/streptavidin The experiment shows that the differences in the unbinding force of the complexes biotin/streptavidin and iminobiotin/streptavidin can be determined by a differential force test.

Principle:

Biotin and iminobiotin are bound to a substrate. Fluorescence-marked streptavidin is bound to the immobilized haptens. A plunger or pillar which is coated with biotin is moved towards the substrate in such a way that the biotin which is bound to the pillar can bind the streptavidin coupled by way of the haptens to the substrate. The pillar is then removed again. To conclude, the procedure involves determining which proportion of the streptavidin was transferred from the iminobiotin of the substrate onto the biotin of the pillar and which proportion of the streptavidin was transferred from the biotin of the substrate onto the biotin of the pillar.

Upon separation of the surfaces, one of the two bonds must become detached. In that situation the ligand remains bound either to the pillar or the substrate, depending on which of the bonds is mechanically more stable.

Implementation:

Coating of the Pillar

A microstructured pillar was made from PDMS (polydimethylsiloxane). In that case the structures consisted of small pillar feet portions measuring about 100×100 μm which were separated by depressions of about 25 μm in width and 1 μm in depth. Bringing the pillar and the substrate into contact presupposes that the buffer therebetween is displaced. That is possible only with extreme difficulty or slowly, when smooth surfaces are involved. The grooves in the pillar ensure that the buffer flows away quickly and guarantees complete contact of the pillar feet portions with the substrate.

A further advantage of the microstructure is that no molecules are “pressed away” by the pillar on the substrate in mirror-image relationship with the grooves in the pillar. The intensity value of the remaining “grids” represents the density of the molecules in front of the pillar and can thus be utilized as a reference value in the evaluation procedure.

To produce the microstructured pillar a composition comprising a 1:10 mixture of silicone elastomer and cross-linking reagent (Sylgard 184, Dow Corning), after multiple degassing, is poured between a suitably microstructured silicon wafer and a smooth plexiglass plate and incubated perpendicularly for 24 hours at ambient temperature. After polymerization the structured surface of the pillar was exposed to an H₂O plasma at 1 mbar in a plasma furnace for 15s. The oxidized surface was incubated with 3% aminosilane (3-aminopropyldimethyl-ethoxysilane; ABCR, Karlsruhe) in 100/% H₂O and 870/% Ethanol for 30 minutes. The silanised surface was washed with very pure water and blown dry with nitrogen.

Attached to the amino groups of the silane was a bifunctional PEG of which one end had a carboxy group activated by NHS, while the other had a biotin group. 20 μl of a solution with 2 mg/100 μl of NHS-PEG-biotin (Shearwater, Huntsville) was incubated under a cover glass for 1 hour on a pillar with an area of 1 cm². It was washed with very pure water and blown dry with nitrogen.

Coating of the Substrate:

A glass object carrier was cleaned by a 100-minute treatment with saturated KOH-ethanol solution. The cleaned surface was incubated with 3% aminosilane (3-aminopropyldimethyl-ethoxysilane; ABCR, Karlsruhe) in 10% H₂O and 87% Ethanol for 30 minutes. The silanised surface was washed with very pure water and blown dry with nitrogen. Attached to the amino groups of the silane was a bifunctional PEG of which one end had a carboxy group activated by NHS, while the other had a t-Boc protected amino group (NHS-PEG NH-tBoc, Shearwater, Huntsville). The tBoc protective group was then split off with trifluoro-acetic acid. NHS-biotin and NHS-iminobiotin (Sigma, St. Louis) were respectively diluted starting from a 50 mM stock solution in DMSO to give a final concentration of 5 mM with PBS (phosphate buffered saline, Sigma). The attachment of biotin or iminobiotin respectively to the amino-functionalized glass substrate was effected from that solution. That was incubated for one hour in a saturated water atmosphere, washed with very pure water and blown dry with nitrogen.

For the purposes of binding the streptavidin AlexaFluor [symbol] -546 conjugate (Molecular Probes, Eugene), a solution was produced, involving a concentration of 0.1 mg/ml in a glycine/NaOH-buffer (pH 10). The substrate was incubated for 20 minutes with the solution, then washed for 5 minutes in that buffer and blown dry with nitrogen.

Pillar Procedure:

The pressing or pillar procedure can be carried out with a simple apparatus as is shown by way of example in FIG. 1. The apparatus comprises a base plate (1), two guide bars (2), a pillar slider or carriage (3), a pillar head pad (4), the pillar head (5) and the pillar pad (6) (see FIG. 7). The base plate, the guide bars and the pillar carriage can be made from metal. The pillar head pad can comprise a foam rubber and the pillar head can comprise plexiglass. The pillar is square and is of an area of one cm² and is 1 mm thick. The pillar pad is of the same dimensions but is smooth on both sides and comprises for example a particularly soft PDMS.

For the pillar pressing operation the substrate is laid on the base plate and the pillar on the pillar pad. Both are covered with buffer (pH 10). The carriage is introduced into the guide bars and moved downwardly by hand until the pillar comes into contact with the substrate. Separation is also effected by hand.

The function of the pillar head pad is to bring the pillar head into an exactly parallel position with respect to the substrate, when applying the pillar. The pillar pad serves to compensate for slight unevenness between the pillar and the substrate.

The surfaces were moved towards each other in such a way that the biotin bound to the pillar can interact with the streptavidin of the substrate. After an incubation time of 30 minutes the surfaces were separated from each other.

Measurement

The pillar and the substrate were scanned with a laser scanner (Perkin Elmer GeneTac LS IV) in respect of the marker Alexa-Fluor [symbol] -546.

Evaluation and Result:

The differing transfer from biotin or iminobiotin to biotin reflects the differing mechanical stability of the streptavidin-hapten complexes. The transfer of biotin onto biotin corresponds to half the actual coupling events, that is to say the coupling number corresponds to double the transfer.

15% of the streptavidin bound to biotin is entrained by the biotin-coated pillar. 30% is transferred from iminobiotin to biotin. On the assumption that the coupling efficiency for both tests is identical and subject to the condition that the density of the iminobiotin and the biotin on the substrate is equal, it can be concluded that the streptavidin-biotin bond is stronger than the streptavidin-iminobiotin bond.

FIGS. 8A and 8B show a representation of the substrate and the pillar after the pillar pressing operation. Measurements with the AFM force spectrometer gave 160 pN±20 pN for the receptor-ligand pair biotin/avidin and 85±15 for iminobiotin/avidin (Florin, E. L., Moy V. T. and Gaub H. E. Science 15, April 1994, Vol. 264, pp 415-417: “Adhesion Forces Between Individual Ligand-Receptor Pairs”). The force comparison between biotin/streptavidin and iminobiotin/streptavidin comes to the same result in qualitative terms.

### EXPERIMENTAL EXAMPLE 2

Force test with reverse pillar pressing using the example of a comparison of the complexes biotin/streptavidin and desthiobiotin/streptavidin

The experiment showed that the differences in the unbinding forces of the complexes biotin/streptavidin and desthiobiotin/streptavidin can be determined by a differential force test with reverse pillar pressing.

Production of Pillar and Substrate:

A microstructured pillar was made from PDMS (polydimethylsiloxane). In that case the structures consisted of small pillar feet portions measuring about 100×100 μm which were separated by depressions of about 25 μm in width and 1 μm in depth. Bringing the pillar and the substrate into contact presupposes that the buffer therebetween is displaced. That is possible only with extreme difficulty or slowly, when smooth surfaces are involved. The grooves in the pillar ensure that the buffer flows away quickly and guarantees complete contact of the pillar feet portions with the substrate.

A further advantage of the microstructure is that no molecules are “pressed away” by the pillar on the substrate in mirror-image relationship with the grooves in the pillar. The intensity value of the remaining “grids” represents the density of the molecules in front of the pillar and can thus be utilized as a reference value in the evaluation procedure.

To produce the 1 mm thick microstructured pillar a composition comprising a 1:10 mixture of silicone elastomer and cross-linking reagent (Sylgard 184, Dow Corning) was poured after multiple degassing between a suitably structured silicon wafer and a smooth plexiglass plate and polymerized perpendicularly for 24 hours at ambient temperature.

The substrate also consisted of 1 mm thick PDMS. However the substrate was not structured. For production thereof the mixture was poured between two perpendicularly disposed plexiglass plates and also incubated for 24 hours at ambient temperature.

Coating of Pillar and Substrate:

The polymerized structured and unstructured PDMS plates were cut to a size of 1 cm². Then the pieces were exposed to an H₂O plasma in a plasma furnace at 2 mbar for 30 s. The oxidized surface was incubated with a solution of 2% aldehyde silane (4-triethoxysilylbutanal, Amchro, Hattersheim, Germany) in 10% H₂O and 88% ethanol for 30 minutes. The silanised surface was washed with ethanol and very pure water and blown dry with nitrogen.

The functionalized substrates and pillars were incubated overnight in PBS (phosphate buffered saline; Sigma, St. Louis, USA) with 2% BSA (bovine serum albumin; Roth, Karlsruhe, Germany). In that case BSA binds with its amino groups to the aldehyde groups of the surface. For stabilization purposes the resulting Schiff's bases were reduced with 1% sodium borohydride for 15 minutes. Then NHS-activated biotin and desthiobiotin (Sigma, St. Louis, USA) was bound to unused amino groups of the BSA. For that purpose, 50 mM stock solutions in DMSO were respectively produced. They were then diluted with PBS to produce a 5 mM reaction solution, in which respect 50 mM EDC (1-ethyl-3-(dimethylamino-propyl)carbiimide; Sigma, St. Louis) and 25 nM NHS(N-hydroxy succinimide; Sigma, St. Louis) were added for activation purposes. The reaction solution was pre-incubated for 15 minutes and then incubated for 30 minutes on the BSA surface. Carboxy groups additionally activated on the BSA were blocked for 2 hours in a 0.1M glycine solution. The pillars and the substrates were washed with very pure water and blown dry with nitrogen.

Depending on the respective pillar composition a fluorescence-marked streptavidin-AlexaFluor [symbol] -647 conjugate (Molecular Probes, Eugene) was bound to the substrate or the pillar. A 0.1 mg/ml concentrated solution in PBS with 0.050/% Tween20 (Sigma, St. Louis, USA) was for that purpose incubated for 20 minutes on the corresponding surface.

Pillar Procedure:

The pressing or pillar procedure can be carried out with a simple apparatus as is shown by way of example in FIG. 7. The apparatus comprises a base plate (1), two guide bars (2), a pillar slider or carriage (3), a pillar head pad (4), the pillar head (5) and the pillar pad (6) (see FIG. 7). The base plate, the guide bars and the pillar carriage can be made from metal. The pillar head pad can comprise a foam rubber and the pillar head can comprise plexiglass. The pillar can be square and of an area of one cm² and 1 mm thick. The pillar pad is of the same dimensions but is smooth on both sides and comprises for example a particularly soft PDMS.

The function of the pillar head pad is to bring the pillar head into an exactly parallel position with respect to the substrate, when applying the pillar. The pillar pad serves to compensate for slight unevenness between the pillar and the substrate.

For the pillar pressing operation the substrate is laid on the base plate and the pillar on the pillar pad. Both are covered with buffer. The carriage is introduced into the guide bars and moved downwardly by hand until the pillar comes into contact with the substrate. Separation is also effected by hand.

The surfaces were moved towards each other in such a way that the free binding partner on the one surface can interact with the complex of streptavidin and the respective other binding partner on the other surface. After an incubation time of 30 minutes the surfaces were separated from each other.

The following reverse procedures were implemented:

| | Procedure | | | |
|---|---|---|---|---|
| | 1 | 2 | 3 | 4 |
| Pillar | Biotin | Desthiobiotin | Biotin | Desthiobiotin |
| | Streptavidin | Streptavidin | | |
| Sub- | Desthiobiotin | Biotin | Streptavidin | Streptavidin |
| strate | | | Desthiobiotin | Biotin |

The attachment of the streptavidin to the substrate or the pillar, with the same surfaces, must give the same result and therefore serves as a control.

The pillar and the substrate were scanned with a laser scanner (GenePix 4000B, Axon Instruments Inc., USA), in respect of the Marker AlexaFluor [symbol] -647.

Evaluation and Result:

Principle:

The reverse implementation permits evaluation which is independent of the coupling efficiency. In this case there can be different densities of binding partners on the first and second surfaces. [This is achieved in that only the maximum possible couplings are taken into consideration, as only those can be shared to the two surfaces]. In the evaluation procedure only the transferred fluorophores are taken into consideration. FIG. 12 shows that the ratio of the binding forces of the various binding complexes B1 and B2 can be calculated from the quotient of the fluorescence intensity which transfers onto surface 1 and that which transfers onto surface 2 (FIG. 12B: number of the fluorophores transferred from the first onto the second surface=2; FIG. 12D: number of the fluorophores transferred from the second onto the first surface=6). In this respect it is only presupposed that, on the surface which has the lower density of binding partners, it is precisely that density that is constant for both partial executions of the reverse implementation. That assumption is permissible here by virtue of the very similar properties of the binding partners BP1 and BP4 to be compared (for example size) and can also be checked by measurement of fluorescence intensity prior to the pillar pressing procedure. This evaluation procedure does not take into consideration the influence of different affinity constants on coupling efficiency.

FIG. 13 shows by way of example the measurement results and the evaluation thereof. As here two spots which were not completely congruent were brought into contact, the overall transfer and therebeside the non-specific transfer can be ascertained in the overlap region of the spots, by means of line scans. The mean values in respect of overall transfer and non-specific transfer were calculated from all rows of the spots, which were measured in 2 independent tests. Specific transfer arises out of the difference of the overall transfer and the non-specific transfer.

The mean values of the reverse procedures of the pressing experiments are shown in the following Tables:

Transfer of streptavidin from the substrate to the pillar:

| Combination | Overall transfer | Non-specific | Specific |
|---|---|---|---|
| Biotin->desthiobiotin | 9600 ± 2500 | 7600 ± 900 | 200 |
| Desthiobiotin->biotin | 23600 ± 5200 | 10200 ± 1600 | 13400 |

Transfer of streptavidin from the pillar to the substrate:

| Combination | Overall transfer | Non-specific | Specific |
|---|---|---|---|
| Biotin->desthiobiotin | 10800 ± 3700 | 7500 ± 1900 | 3300 |
| Desthiobiotin->biotin | 23200 ± 700 | 6900 ± 1100 | 16300 |

Within the limits of measuring accuracy both tests afford the same result. The quotient of the transfer to desthiobiotin and the transfer to biotin is on average 0.18. Clear qualitative information about the binding forces is possible on the basis of that relationship: the reverse pillar pressing operation shows that the binding force between streptavidin and desthiobiotin is weaker than the binding force between streptavidin and biotin. As streptavidin/biotin/desthiobiotin is a complex system in which multiple bonds to a surface are possible, quantitative information relating to the relationship of the binding forces is only limitedly possible.

### EXPERIMENTAL EXAMPLE 3

Force test for the Comparison of Two DNA-Duplexes

The experiment shows that the unbinding forces of two DNA-duplexes can be compared by a differential force test. In this Example duplex 1 involves a 20 base pairs long double strand while duplex 2 involves a 30 base pairs long double strand.

Principle:

Oligonucleotide 1 (=oligo1) is terminally bound to a substrate. Oligonucleotide 2 (=oligo2) is hybridized with oligo1, a sample complex then being formed. Oligonucleotide 3 (═Oligo3) is hybridized with oligo2, with a reference complex being formed. Oligo2 and oligo3 are marked with different fluorophores. Oligo3 is additionally marked with a biotin. A pillar which is coated with streptavidin is pressed onto the substrate with the three hybridized oligos. That gives rise to binding of the biotin from oligo3 to the streptavidin of the pillar. The pillar is removed, in which case in an interlinkage of oligo1 with oligo2 and oligo3 either tearing of the sample complex or tearing of the reference complex occurs.

The sample complex is a DNA-duplex of 20 bp. The reference complex comprises a DNA-duplex of 30 bp, of which 20 are identical to those of the sample complex. As a reference a second experiment is carried out, in which the sample and the reference complexes are 20 bp long and have the same GC-content.

As the efficiency with which coupling of the interlinkages pre-formed on the substrate is effected by way of the biotin/streptavidin bond, can be only limitedly monitored in the pillar pressing operation, it is not possible in the force test to limit the procedure to only marking oligo2 in order to calculate the ratio of the unbinding forces of the sample and the reference complex. A second marking in respect of oligo3 is required. If on the basis of the two markings the procedure involves determining how much oligo2 was enriched in relation to oligo3 on the pillar or depleted on the substrate, that affords a measurement of whether oligo2 has a greater unbinding force in relation to oligo1 or oligo3.

It is to be noted that, in the event of fluorescence marking of the oligos, no falsification of the measurement result occurs by virtue of fluorescence resonance transfer (FRET) between the markers. As a small amount of FRET between the markers of an interlinkage is often inevitable, attention is to be paid to the markers within a interlinkage in the experiment and the reference experiment being at the same spacing from each other.

Experiment: 20 bp against 30 bp (=Experiment 2a)

```
5′NH2-AAAAAAAAAA TCTCCGGCTTTACGGCGTAT oligo1
                                  ||||||||||||||||||||
                      3′Cy3-AGAGGCCGAAATGCCGCATA
TTGGGGAGCAATGCTAATAGTT TCCCTGAAAGTCGTCTCTCAGACCCTCGTT oligo2a
                                            |||||||||||||||||||||||||| ||||||
                  oligo3 5″Cy5-AGGGACTTTCAGCAGAGAGTCTGGGAGCAA AAAAAAAAAA-Bio
```

Experiment: 20 bp against 20 bp (=Experiment 2b)

```
5′NH2-AAAAAAAAAA TCTCCGGCTTTACGGCGTAT oligo1
                                  ||||||||||||||||||||
                      3′Cy3-AGAGGCCGAAATGCCGCATA
TTGGGGAGCAATGCTAATAGTT TCCCTGAAAGTCGTCTCTCA oligo2b
                                          ||||||||||||||||||||
              oligo3 5′ Cy5-AGGGACTTTCAGCAGAGAGTCTGGGAGCAA AAAAAAAAAA-Bio
```

Implementation:

1. Coating of the Substrate:

The substrate used was glass object carriers functionalized with aldehyde groups (Telechem, Atlanta: Superaldehyde Slides). Polyethylene glycol (PEG) was covalently bound to the glass surface as passivation against adsorption of the streptavidin. For that purpose 2 mg of bifunctional PEG (Shearwater, Huntsville; molecular weight=3400 g/mol) with respectively a terminal amino group and a terminal carboxy group was dissolved in 1001 PBS (Phosphate Saline Buffer, Sigma, St. Louis) and incubated under a cover glass for 2 hours on the object carrier. The Schiff's bases resulting from the reaction of the amino groups with the aldehyde groups were reduced for 5 minutes with a 10/% NaBH₄ solution.

Thereupon the substrate was washed with very pure water and blown dry with an oil-free nitrogen jet.

2. Binding of oligo1:

Oligo1: 5′ NH2-AAAAAAAAAA TCTCCGGCTTTACGGCGTAT (SEQ ID NO:1)

Oligo1 has at the 5′-end an amino marker and a spacer of 10 adenines. The further 20 bases form with oligo2 the sample complex. Several drops of 1 μl respectively of a mixture of 25 μM oligo1 with 5 mg/ml ECD (1-ethyl-3-(3-dimethylamino-propyl)carbiimide; Sigma, St. Louis) and 5 mg/ml NHS(N-hydroxy-succinimide; Sigma, St. Louis) in PBS (Phosphate Saline Buffer; Sigma, St. Louis) were spotted onto the coated substrate. The substrate was incubated in a saturated H₂O atmosphere for 1 hour, washed with 0.20/o SDS (sodium dodecylsulfate; Sigma, St. Louis), rinsed with very pure water and blown dry with nitrogen.

3. Hybridization:

| | |
|---|---|
| Oligo2a: | |
| 5′TTGCTCCCAGACTCTCTGCTGAAAGTCCCTTTGATAATCGTAACGAGGGGTTATACGCCGTAAAGCCCGGAGA-Cy3 | (SEQ ID NO:2) |
| Oligo2b: | |
| 5′ACTCTCTGCTGAAAGTCCCTTTGATAATCGTAACGAGGGGTTATACGCCGTAAAGCCGGAGA-Cy3 | (SEQ ID NO:3) |
| Oligo3: | |
| 5′Cy5-AGGGACTITCAGCAGAGAGTCTGGGAGCAA AAAAAAAAAA-BIO | (SEQ ID NO:4) |

The oligos 2a and 2b have a Cy3 [symbol] -marker (cyanine3, Amersham-Pharmacia Biotech) at the 5′-end. Oligo3 has a Cy5 [symbol] -marker (cyanine5, Amersham-Pharmacia) at the 5′-end (all oligos from metabion, Martinsried).

5 μl of a mixture of oligo2a (1 μM) and oligo3 (2 μM) in a citrate buffer (pH=7.2) with 750 mM NaCl was put under a cover glass over the spots of the bound oligo1. The hybridization composition was incubated in a saturated H₂O atmosphere at 80° C. for 15 minutes and then cooled down to ambient temperature. The substrate was washed once with 15 mM NaCl/0.2% SDS and a second time with 15 mM NaCl at ambient temperature. The procedure then involved rinsing with very pure water and blowing it dry with nitrogen. The procedure was likewise in the reference experiment, with oligo2a being replaced by oligo2b.

4. Coating of the Pillar:

A 1 mm thick microstructured pillar was made from PDMS (polydimethylsiloxane). The structure in that case consisted of pillar foot portions of about 100×100 μm which were separated by depressions of about 25 μm in width and one μm in depth. For that purpose a composition comprising a 1:10 mixture of silicone elastomer and cross-linking reagent (Sylgard 184, Dow Corning), after multiple degassing, was poured between a suitably structured silicon wafer and a smooth plexiglass plate and incubated for 24 hours at ambient temperature. After polymerization the structured surface of the pillar was exposed to an H₂O plasma at 1 mbar in a plasma furnace for 15 seconds.

The oxidized surface was incubated with 30/% aminosilane (3-aminopropyldimethyl-ethoxysilane; ABCR, Karlsruhe) in 10%/o H₂O and 870/o ethanol for 30 minutes. The silanised surface was washed firstly with ethanol and then with very pure water and blown dry with nitrogen. A bifunctional PEG was bound to the amino groups of the silane, one end of the PEG having an NHS-activated carboxy group and the other a biotin group. 20 μl of a solution with 20 mg/ml NHS-PEG-biotin (Shearwater, Huntsville) was incubated under a cover glass for 1 hour on a pillar with a surface area of 1 cm². The procedure then involved washing with very pure water and blowing dry with nitrogen. 0.1 mg/ml of streptavidin (Sigma) in PBS was put onto the surface which was now biotinylised and incubated for 30 minutes. Washing was effected with very pure water, followed by blowing dry with nitrogen.

5. Pillar Pressing:

A freshly prepared pillar and a substrate were pressed with a solution of 15 mM NaCl under a pressure of 400 g/cm² onto the spots with the bound oligos (oligo1+oligo2+oligo3). After 30 minutes the pillar was very slowly lifted off. The substrate and the pillar were washed with very pure water and blown dry with nitrogen.

6. The pillar and the substrate were scanned with a two-color laser scanner (Perkin Elmer GeneTac LS IV) in respect of the markers Cy3 and Cy5. In order to ensure comparability of the measurement results the pillars of the experiment and the reference experiment and the substrates of the experiment and the reference experiment were scanned with equal laser intensities.

Evaluation:

The pillar and the substrate were evaluated for each experiment.

Evaluation of the Substrates:

The differences ΔCy3_{Unt} and ΔCy5_{Unt} were formed by subtraction of the intensities of the pressed surfaces from the intensities of the non-pressed grooves. The quotient Q_{A Unt}=ΔCy3_{Unt}/ΔCy5_{Unt} was formed from the two differences for Experiment A (oligo2a) and the quotient Q_{B Unt}=ΔCy3_{Unt}/ΔCy5_{Unt} was formed from the differences for Experiment B (oligo2b). The quotient Q_{Unt}=Q_{A Unt}/Q_{B Unt} was formed as a measurement in respect of the difference in depletion of oligo2a and oligo2b respectively on the substrate.

Evaluation of the Pillars:

The differences ΔCy3_{St} and ΔCy5ₛₜ were formed by subtraction of the intensities of the grooves to which no oligos were transferred from the intensities of the pillar surfaces. The quotient Q_{A St}=ΔCy3_{St}/ΔCy5_{St} was formed from the two differences for Experiment A (oligo2a) while the quotient Q_{B St}=ΔCy3_{St}/ΔCy5_{St} was formed from the differences for Experiment B (oligo2b). The quotient Q_{St}=Q_{A St}/Q_{B St} was formed as a measurement in respect of the difference in enrichment of oligo2a and oligo2b respectively on the substrate.

Result

For the substrate of Experiment 2b the procedure gave the value Q_{B Unt}=0.36±0.08 and for the substrate of 2a Q_{A Unt}=0.69±0.09. That gave Q_{Unt}=Q_{A Unt}/Q_{B Unt}=1.92.

For the pillar of 2b the procedure gave Q_{B St}=0.61+0.08, and for the pillar of 2a Q_{A St}=1.20+0.08. That gave Q_{St}=Q_{A St}/Q_{B St}=1.97.

Experiment 2a involved a force comparison of a 30 bp duplex on the side of the pillar and a 20 bp duplex on the side of the substrate. If the result for 2a (Q_{A Unt}=0.69, Q_{A St}=1.2) is considered in isolation, it is not possible to have any information as to whether one of the two duplexes was more stable or which of the two, when separating the pillar from the substrate, tore more frequently than the other. Such information from Experiment 2a alone would only be possible if the actual substance amounts of Cy3 and Cy5 could be calculated from the measured levels of fluorescence intensity. As the present Experiment does not involve corresponding calibration values, for the force comparison of the 30 bp duplex with the 20 bp duplex in Experiment 2a the force comparison of the 20 bp duplex with a further 20 bp duplex in Experiment 2b is to be used as a reference.

The quotients from Experiment 2a and reference experiment 2b give for the substrate Q_{Unt}=Q_{A Unt}/Q_{B Unt}=1.92 and for the pillar Q_{St}=Q_{A St}/Q_{B St}=1.97. That means that on the substrate 2a approximately twice as much Cy3-marked oligo was pressed away as in 2b and that approximately twice as much Cy3-marked oligo was transferred onto the pillar as in 2b.

In a measurement procedure in which the same number of Cy5- and Cy3-fluorophores would have the same levels of fluorescence intensity, a Q_{B Unt}=0.5 and a Q_{B St}=0.5 would be expected in the reference experiment 2b, by virtue of the identical stability of the two 20 bp duplexes. The Q_{B Unt}=0.36, which is ascertained here, is too small by the factor 1.39 in relation to the result to be expected in a calibrated measurement procedure. If Q_{B Unt} is corrected by that factor to 0.5, that gives for the correction of Experiment 2a:

Q_{A Unt corr}.=Q_{A Unt}×1.39=0.69×1.39=0.96.

For the PDMS pillar, by virtue of the specific fluorescence properties of the material, it is necessary to calculate a particular correction factor for same. Similarly to the substrate that gives:

Q_{BSt corr.}=0.5=Q_{B St}×0.82

Q_{A St corr.}=1.20×0.82=0.98

It can be concluded therefrom that in all interlinkages formed 97% of all sample complexes (20 bp) tear and only 3% of all reference complexes (30 bp).

FIGS. 9A and 9B show a view of the substrate and the pillar after the pressing operation in Experiment 2a. FIGS. 9C and 9D show a view of the substrate or the pillar after the pressing operation in Experiment 2b. In each case only the color or dye Cy3 is shown.

FIGS. 10A and 10C show the result of the substrate after force comparison with oligonucleotide 2a or 2b respectively. FIGS. 10B and 10D correspondingly show the result for the pillars. This involves portions of fluorescence profiles. The progression of the profiles is indicated by the arrow in FIG. 9C. The maxima of the graphs 10A and 10C correspond to the bright grid lines which represent non-pressed regions of the substrate. The minima which are between the peaks correspond to the dark squares from which oligos were pressed away as a consequence of the contact with the streptavidin bound on the pillar foot portions. The minima of the graphs in 10B and 10D correspond to the dark grid lines on the pillars, to which no fluorophore was transferred. The maxima correspond to the bright square to which oligos were transferred onto the pillar, as a consequence of the contact with the substrate.

Q_{St} and Q_{Unt} showed that the duplex between oligo2a and oligo 3 has a unbinding force which is at least 500/% higher than the duplex between oligo 2b and oligo3.

1. A method of characterizing and/or identifying a binding complex, comprising the steps:
preparing a first binding partner and a conjugate of a second and a third binding partner and preparing a fourth binding partner,
forming an interlinkage of the binding partners, wherein the first binding partner with the second binding partner forms a sample complex and the third binding partner with the fourth binding partner forms a reference complex,
applying a force to the interlinkage which results in unbinding of the sample complex or the reference complex, and
determining which of the two binding complexes was unbound.

2. A method as set forth in claim 1 wherein firstly the conjugate is prepared from the second and third binding partners and then the sample complex and/or the reference complex is formed.

3. A method as set forth in claim 1 wherein firstly the sample complex and/or the reference complex is formed and then the conjugate is prepared by connecting the second and third binding partners.

4. A method as set forth in claim 3 comprising the steps of immobilizing the first binding partner to a first holding means, immobilizing the fourth binding partner to a second holding means, producing the sample complex by bringing the immobilized first binding partner into contact with the second binding partner, producing the reference complex by bringing the immobilized fourth binding partner into contact with the third binding partner, and moving the first and second holding means towards each other, in which case the second and third binding partners can come into interaction with each other.

5. A method as set forth in one of claims 1 through 4 wherein the first binding partner and the fourth binding partner are identical.

6. A method as set forth in one of claims 1 through 5 wherein the first and second binding partners are a ligand and a receptor which specifically bind to each other.

7. A method as set forth in one of claims 1 through 5 wherein the sample complex is based on a non-specific interaction.

8. A method as set forth in claims 1 through 7 wherein the reference complex is based on a specific or a non-specific interaction.

9. A method as set forth in claims 1 through 8 wherein at least one of the binding partners preferably of the sample complex is a body.

10. A method as set forth in one of the preceding claims wherein at least one of the binding partners of the sample complex is a biomolecule.

11. A method as set forth in one of the preceding claims wherein the first and/or fourth binding partners are fixed to a first and a second holding means respectively, which preferably each have a respective body.

12. A method as set forth in one of claims 9 through 11 wherein at least one body has a macroscopically large surface to which preferably a plurality of sample complexes and/or reference complexes can be connected.

13. A method as set forth in one of claims 9 through 12 wherein at least one body is nanoscopically small, preferably selected from a group which includes particles, magnetic particles, paramagnetic particles, diamagnetic particles, colloids, molecules, charged molecules, polymers and multiply charged polymers.

14. A method as set forth in one of the preceding claims wherein the force for unbinding the interlinkage is applied by a macroscopic tension.

15. A method as set forth in one of the preceding claims wherein the force is applied by a magnetic field.

16. A method as set forth in one of the preceding claims wherein the force is applied by a hydrodynamic flow.

17. A method as set forth in one of the preceding claims wherein the force is applied by coupling in sound waves, preferably ultrasonic waves.

18. A method as set forth in one of the preceding claims wherein the force is built up by applying electrostatic forces.

19. A method as set forth in one of the preceding claims wherein the force is applied by molecular conformational changes of suspension means and/or the connections.

20. A method as set forth in one of the preceding claims wherein a force is applied while maintaining a given force rate.

21. A method as set forth in claim 20 wherein setting of the force rate is effected by way of the pulling speed with which the holding means are separated.

22. A method as set forth in claim 20 or claim 21 wherein setting of the force rate is effected by way of the spring constant of the interlinkage with the bonds thereof.

23. A method as set forth in claim 22 wherein setting of the spring constant is effected by way of a variation in the length of a polymer of which the bonds of the ligands or the connection which connects the receptors consists.

24. A method as set forth in one of the preceding claims wherein a sample complex and/or a reference complex has at least one constituent selected from a group including low-molecular substances, polymers, proteins, antibodies, antigens, haptens, natural or synthetic nucleic acids, particles, viruses, phages, cells, cell constituents and/or complex-forming substances such as chelating agents.

25. A method as set forth in one of claims 1 through 24 comprising the steps of immobilizing the first binding partner to a first holding means, immobilizing the fourth binding partner to a second holding means, preparing a conjugate of the second and third binding partners, which represents the sample, wherein the second binding partner can bind to the first binding partner and the third binding partner can bind to the fourth binding partner, and moving the first and second holding means towards each other, in which case the binding partners of the sample can come into interaction with the other two associated binding partners.

26. A method as set forth in one of claims 1 through 24 comprising the steps of immobilizing the first binding partner which represents the sample to a first holding means, immobilizing the fourth binding partner to a second holding means, preparing a conjugate of the second and third binding partners, wherein the second binding partner can bind to the sample and the third binding partner can bind to the fourth binding partner, and moving the first and second holding means towards each other, wherein the associated binding partners can come into interaction.

27. A method as set forth in one of the preceding claims including the step of marking the sample complex and/or the reference complex, preferably at least of one binding partner, and indirect or direct detection of the unbinding location which occurs after application of the force.

28. A method as set forth in claim 27 wherein the conjugate of the second and third binding partners, the second binding partner or the third binding partner are provided with a first marker, and the first or the fourth binding partners are provided with a second marker which is different from the first marker.

29. A method as set forth in claim 28 wherein detection of the unbinding location is effected by ascertaining the amount of first marker which is bound to one of the holding means, ascertaining the amount of second marker which is bound to the same holding means, and comparing the ascertained values to each other and/or relating them to each other.

30. A method as set forth in one of claims 1 through 3, 5 through 24 or 27 through 29 characterized in that firstly the interlinkage which includes the first, second, third and fourth binding partners is formed on a first holding means and then in a second step coupling to a second holding means is effected.

31. A method as set forth in one of claims 1 though 3 or 5 through 29 characterized in that
(i) the first binding partner BP1 and the conjugate including the second binding partner BP2 and the third binding partner BP3 are bound on a first holding means,
the fourth binding partner BP4 is immobilized on a second holding means,
the two holding means are moved towards each other so that the third binding partner BP3 and the fourth binding partner BP4 can bind to each other, or
(ii) the fourth binding partner BP4 and the conjugate including the second binding partner BP2 and the third binding partner BP3 are bound on the second holding means,
the first binding partner BP1 is immobilized on the first holding means, and
the two holding means are moved towards each other, so that the second binding partner BP2 and the first binding partner BP1 can bind to each other.

32. A method as set forth in one of claims 27 through 31 wherein at least one of the binding partners includes a nucleic acid, in particular DNA.

33. A method as set forth in one of claims 27 through 32 wherein at least two of the binding partners include a natural or synthetic nucleic acid.

34. A method as set forth in one of claims 27 through 33 when marking is effected by fluorescing molecules.

35. A method as set forth in claim 34 wherein a binding partner of a binding complex is provided with a first fluorophore and the second binding partner is provided with a second fluorophore, between which fluorescence resonance transfer (FRET) takes place.

36. A method as set forth in claim 34 wherein a binding partner of a binding complex is provided with a fluorophore and the second binding partner is provided with a molecule which quenches the fluorescence of the fluorophore.

37. A method as set forth in one of claims 27 through 33 wherein the marking involves fluorescing nanoscopic semiconductor particles (quantum dots).

38. A method as set forth in one of claims 27 through 33 which involve radioactive marking.

39. A method as set forth in one of claims 27 through 33 wherein the marker involves an enzyme or an affinity marker to which an enzyme which by a reaction develops a signal substance can bind.

40. A method as set forth in claim 39 wherein the activation of an enzyme which develops a detectable signal is coupled to the unbinding of a complex.

41. A method as set forth in one of claims 27 through 33 wherein the marker involves a molecule of electroluminescence, an electrochemically detectable molecule or a mass marker which can be detected by mass spectroscopy.

42. A method as set forth in one of claims 1 through 41 wherein the method is implemented on many similar interlinkages.

43. A method as set forth in claim 42 wherein only one kind of interlinkage is tested at only one force rate, wherein the interlinkage always includes the same sample complex and always the same reference complex.

44. A method as set forth in claim 42 wherein only one kind of interlinkage is tested with different force rates, wherein the interlinkage always includes the same sample complex and always the same reference complex.

45. A method as set forth in claim 42 wherein various interlinkages are tested at only force rate, wherein the interlinkages always include the same sample complex but varying reference complexes with different unbinding forces.

46. A method as set forth in claim 42 wherein various interlinkages are tested at various force rates, wherein the interlinkages always include the same sample complex but varying reference complexes with different unbinding forces.

47. A method as set forth in one of claims 44 through 46 wherein the interlinkages are serially tested on only one procedure.

48. A method as set forth in one of claims 44 through 46 wherein the various interlinkages or force rates are tested preferably parallel in separate procedures.

49. A method as set forth in one of the preceding claims characterized in that in addition the coupling number and/or coupling efficiency, namely the quotient of the number of couplings actually formed and the number of maximum possible couplings, is at least approximately determined, and optionally the coupling number and/or the coupling efficiency is taken into consideration in determining whether the sample complex or the reference complex was unbound.

50. A method as set forth in claim 49 characterized in that
(i) in a first step (=force comparison) the amount of conjugate including the second binding partner BP2 and the third binding partner BP3, which was transferred after the separation operation from a first holding means onto a second holding means is determined, and/or the amount of conjugate including the second binding partner BP2 and the third binding partner BP3, which after the separation operation was not transferred from the first holding means onto the second holding means is determined,
wherein the first binding partner BP1 is different from the fourth binding partner BP4, and
(ii) in a second step (=self-comparison), to determine the coupling efficiency, the amount of conjugate including the second binding partner BP2 and the third binding partner BP3, which after the separation operation was transferred from a first holding means onto a second holding means, is determined, and/or the amount of conjugate including the second binding partner BP2 and the third binding partner BP3, which after the separation operation was not transferred from the first holding means onto the second holding means, is determined,
wherein the first binding partner BP1 is also used as the fourth binding partner BP4, or the fourth binding partner BP4 is also used as the first binding partner BP1, so that the first binding partner BP1 and the fourth binding partner BP4 in the second step are identical and the second binding partner BP2 is substantially identical to the third binding partner BP3.

51. A method as set forth in claim 49 characterized in that
(i) the first binding partner BP1 and the conjugate including the second binding partner BP2 and the third binding partner BP3 are bound on a first holding means,
the fourth binding partner BP4 is immobilized on a second holding means,
the two holding means are moved towards each other so that the third binding partner BP3 and the fourth binding partner BP4 can bind to each other, the amount of conjugate including the second binding partner BP2 and the third binding partner BP3, which after the separation operation was transferred from the first holding means onto the second holding means, is determined, and/or the amount of conjugate including the second binding partner BP2 and the third binding partner BP3, which after the separation operation was not transferred from the first holding means onto the second holding means, is determined,
(ii) the fourth binding partner BP4 and the conjugate including the second binding partner BP2 and the third binding partner BP3 are bound on the second holding means,
the first binding partner BP1 is immobilized on the first holding means,
the two holding means are moved towards each other, so that the second binding partner BP2 and the first binding partner BP1 can bind to each other,
the amount of conjugate including the second binding partner BP2 and the third binding partner BP3, which after the separation operation was transferred from the second holding means onto the first holding means, is determined, and/or the amount of conjugate including the second binding partner BP2 and the third binding partner BP3, which after the separation operation was not transferred from the second holding means onto the first holding means, is determined.

52. Apparatus for characterizing and/or identifying a binding complex, comprising:
a first binding partner and a conjugate of a second and a third binding partner and a fourth binding partner,
a means for interlinkage of the binding partners, wherein the first binding partner with the second binding partner forms a sample complex and the third binding partner with the fourth binding partner forms a reference complex,
a means for applying a force to the interlinkage which results in unbinding of the sample complex or the reference complex, and
a means for determining which of the two binding complexes was unbound.

53. Apparatus as set forth in claim 52 wherein firstly the conjugate is prepared from the second and third binding partners and then the sample complex and/or the reference complex is formed.

54. Apparatus as set forth in claim 52 wherein firstly the sample complex and/or the reference complex is formed and then the conjugate is prepared by connecting the second and third binding partners.

55. Apparatus as set forth in claim 54 wherein the first binding partner is immobilized to a first holding means, the fourth binding partner is immobilized to a second holding means, the second binding partner with the first binding partner forms the sample complex, and the third binding partner with the fourth binding partner forms the reference complex, and including a means for moving the first and second holding means towards each other, in which case the second and third binding partners can come into interaction.

56. Apparatus as set forth in one of claims 52 through 55 wherein the first binding partner and the fourth binding partner are identical.

57. Apparatus as set forth in one of claims 52 through 56 wherein the first and second binding partners are a ligand and a receptor which specifically bind to each other.

58. Apparatus as set forth in one of claims 52 through 56 wherein the sample complex is based on a non-specific interaction.

59. Apparatus as set forth in claims 52 through 58 wherein the reference complex is based on a specific or a non-specific interaction.

60. Apparatus as set forth in claim 52 through 59 wherein at least one of the binding partners preferably of the sample complex is a body.

61. Apparatus as set forth in one of claims 52 through 60 wherein at least one of the binding partners of the sample complex is a biomolecule.

62. Apparatus as set forth in one of claims 52 through 61 wherein the first and/or fourth binding partners are fixed to a first and a second holding means respectively, which preferably each have a respective body.

63. Apparatus as set forth in one of claims 60 through 62 wherein at least one body has a macroscopically large surface to which preferably a plurality of sample complexes and/or reference complexes can be connected.

64. Apparatus as set forth in one of claims 60 through 63 wherein at least one body is nanoscopically small, preferably selected from a group which includes particles, magnetic particles, paramagnetic particles, diamagnetic particles, colloids, molecules, charged molecules, polymers and multiply charged polymers.

65. Apparatus as set forth in one of claims 52 through 64 wherein the force means for unbinding of the interlinkage has a means for applying a macroscopic tension.

66. Apparatus as set forth in one of claims 52 through 65 comprising a means for applying magnetic forces.

67. Apparatus as set forth in one of claims 52 through 66 comprising a means for applying hydrodynamic forces.

68. Apparatus as set forth in one of claims 52 through 67 comprising a means for coupling in sound waves, preferably ultrasonic waves.

69. Apparatus as set forth in one of claims 52 through 68 comprising a means for applying electrostatic forces.

70. Apparatus as set forth in one of claims 52 through 69 comprising a means for producing molecular conformational changes of suspension means and/or the connections.

71. Apparatus as set forth in one of claims 52 through 70 wherein a force is applied while maintaining a given force rate.

72. Apparatus as set forth in claim 71 comprising a means for setting the force rate, preferably the pulling speed, with which the holding means are separated.

73. Apparatus as set forth in claim 71 or claim 72 wherein the force rate can be set by way of the spring constant of the interlinkage with the bonds thereof.

74. Apparatus as set forth in claim 73 wherein the spring constant can be set by way of the variation in the length of a polymer of which the bonds of the ligand or the connection which connects the receptors consist.

75. Apparatus as set forth in one of claims 52 through 74 wherein a sample complex and/or a reference complex has at least one constituent selected from a group including low-molecular substances, polymers, proteins, antibodies, antigens, haptens, natural or synthetic nucleic acids, particles, viruses, phages, cells, cell constituents and/or complex-forming substances such as chelating agents.

76. Apparatus as set forth in one of claims 52 through 75 wherein the first binding partner is immobilized to a first holding means, the fourth binding partner is immobilized to a second holding means, a conjugate comprises the second and third binding partners, representing a sample, wherein the second binding partner can bind to the first binding partner and the third binding partner can bind to the fourth binding partner, and including a means for moving the first and second holding means towards each other, wherein the binding partners of the sample can come into interaction with the other two associated binding partners.

77. Apparatus as set forth in one of claims 52 through 75 wherein the first binding partner which represents the sample is immobilized to a first holding means, the fourth binding partner is immobilized to a second holding means, a conjugate comprises the second and the third binding partners, wherein the second binding partner can bind to the sample and the third binding partner can bind to the fourth binding partner, and including a means for moving the first and second holding means towards each other, wherein the associated binding partners can come into interaction.

78. Apparatus as set forth in one of claims 52 through 77 comprising a marking on the sample complex and/or the reference complex, preferably at least one binding partner, and a means for indirect or direct detection of the unbinding location which occurs after application of the force.

79. Apparatus as set forth in claim 78 comprising a first marker on the conjugate of the second and third binding partners and a second marker on the first or fourth binding partner, wherein the second marker is different from the first marker.

80. Apparatus as set forth in claim 78 or claim 79 wherein marking is effected by fluorescent molecules.

81. Apparatus as set forth in claim 80 wherein a binding partner of a binding complex is provided with a first fluorophore and the second binding partner is provided with a second fluorophore, between which fluorescence resonance transfer (FRET) takes place.

82. Apparatus as set forth in claim 80 wherein a binding partner of a binding complex is provided with a fluorophore and the second binding partner is provided with a molecule which quenches the fluorescence of the fluorophore.

83. Apparatus as set forth in claim 78 or claim 79 wherein the marking involves fluorescing nanoscopic semiconductor particles (quantum dots).

84. Apparatus as set forth in claim 78 or claim 79 wherein the marking is radioactive.

85. Apparatus as set forth in claim 78 or claim 79 wherein the marking has an enzyme or an affinity marker to which an enzyme which develops a signal substance by a reaction can bind.

86. Apparatus as set forth in claim 85 wherein activation of an enzyme which develops a detectable signal is coupled to the unbinding of a complex.

87. Apparatus as set forth in claim 78 or claim 79 wherein the marking involves a molecule of electroluminescence, an electrochemically detectable molecule or a mass marking which can be detected by mass spectroscopy.

88. Apparatus as set forth in one of claims 52 through 87 having many similar interlinkages at which the measuring operation is carried out.

89. Apparatus as set forth in claim 88 wherein only one kind of interlinkage is tested at only one force rate, wherein the interlinkage always includes the same sample complex and always the same reference complex.

90. Apparatus as set forth in claim 88 wherein only one kind of interlinkage is tested at various force rates, wherein the interlinkage always includes the same sample complex and always the same reference complex.

91. Apparatus as set forth in claim 88 wherein various interlinkages are tested at only one force rate, wherein the interlinkages always include the same sample complex but varying reference complexes with varying unbinding forces.

92. Apparatus as set forth in claim 88 wherein various interlinkages are tested at various force rates, wherein the interlinkages always include the same sample complex but varying reference complexes with varying unbinding forces.

93. Apparatus as set forth in one of claims 90 through 92 wherein the interlinkages are tested serially on only one procedure.

94. Apparatus as set forth in one of claims 90 through 92 wherein the various interlinkages or force rates are tested preferably in parallel in separate procedures.

95. Apparatus as set forth in one of claims 52 through 94 wherein the unbinding forces of the reference complexes are so selected that the unbinding force of the sample complex can be approximately determined.

96. A kit for identifying a binding complex for carrying out a method as set forth in at least one of claims 1 through 51.

97. A kit for identifying a binding complex having the means as set forth in at least one of claims 52 through 95.


SEQUENCE LISTING (4 sequences)

SEQ ID NO: 1 - DNA, 30 bp, Artificial Sequence
  Description of Artificial Sequence Oligonucleotide
aaaaaaaaaatctccggctttacggcgtat

SEQ ID NO: 2 - DNA, 72 bp, Artificial Sequence
  Description of Artificial Sequence Oligonucleotide
ttgctcccagactctctgctgaaagtccctttgataatcgtaacgaggggttatacgccgtaaagccggaga

SEQ ID NO: 3 - DNA, 62 bp, Artificial Sequence
  Description of Artificial Sequence Oligonucleotide
actctctgctgaaagtccctttgataatcgtaacgaggggttatacgccgtaaagccggaga

SEQ ID NO: 4 - DNA, 40 bp, Artificial Sequence
  Description of Artificial Sequence Oligonucleotide
agggactttcagcagagagtctgggagcaaaaaaaaaaaa